(19) United States
(12) Patent Application Publication
(10) Pub. No.: US 2021/0322464 A1
(43) Pub. Date: Oct. 21, 2021

(54) METHOD OF TREATING AND PREVENTING CORONAVIRUS DISEASE 19 (COVID-19) USING A SELENIUM ADMINISTRATION

(71) Applicant: Mohamed Samir Elsayed Ghoweba, Longview, TX (US)

(72) Inventor: Mohamed Samir Elsayed Ghoweba, Longview, TX (US)

(21) Appl. No.: 17/235,592

(22) Filed: Apr. 20, 2021

Related U.S. Application Data
(60) Provisional application No. 63/012,502, filed on Apr. 20, 2020.

(51) Int. Cl. A61K 33/04; A61P 31/14; A61K 9/00; A61K 31/573; A61K 31/7048; A61K 31/546; A61K 31/7076; A61K 35/16

(52) U.S. Cl. CPC A61K 33/04; A61P 31/14; A61K 9/0019; A61K 35/16; A61K 31/7048; A61K 31/546; A61K 31/7076; A61K 31/573

(57) ABSTRACT

The present invention provides methods for treatment and prevention of Coronavirus Disease 2019 (COVID-19) and any clinical presentations associated with it including Acute Respiratory Distress Syndrome (ARDS), Severe Inflammatory Response Syndrome (SIRS), and/or any state corresponding to a severe acute attack of an inflammatory pathology causing an exacerbation of cytokine storm associated with unregulated oxidative, endoplasmic reticulum, and inflammatory stress mediated cytotoxic effects following the infection with the novel Severe Acute Respiratory Syndrome Coronavirus-2 (SARS-CoV-2) with at least a pharmacologically acceptable trace element, Selenium (Se) containing molecule, or compound, or drug, or injection, or intravenous infusion. This is achieved by administering to a subject, a therapeutically effective amount of at least a pharmacologically acceptable molecule containing Se at an initial high dose followed by reduced, continuous dosing in subsequent treatment so as to employ its antioxidant, cytokine-modulating, antiviral, immune-enhancing, anti-apoptotic, and anticoagulant properties for the treatment and prevention of COVID-19.

### CROSS REFERENCE TO RELATED APPLICATIONS

The current application claims benefit of the U.S. Provisional Patent Application 63/012,502 filed Apr. 20, 2020.

### FIELD OF THE INVENTION

The present invention relates generally to the use of at least a molecule containing Selenium (Se) for a method of treating patients suffering from Coronavirus Disease 2019 (COVID-19) and its associated Acute Respiratory Distress Syndrome (ARDS) and Severe Inflammatory Response Syndrome (SIRS), and/or any state corresponding to a severe acute attack of an inflammatory pathology causing an exacerbation of cytokine release as well as preventing COVID-19 related to the novel Severe Acute Respiratory Syndrome Coronavirus-2 (SARS-CoV-2). More specifically, the present invention relates to the utilization of the antioxidant, cytokine-modulating, antiviral, immune-enhancing, anti-apoptotic, and anticoagulant properties of Selenium and its compounds for the treatment and prevention of COVID-19 and the method of implementing it. The invention is applicable in human and veterinary medicine.

### BACKGROUND OF THE INVENTION

COVID-19 is a respiratory illness that is caused by the novel SARS-CoV-2. Illness severity can widely range from mild, moderate, severe featuring pneumonia, to critical. Despite ongoing extensive research to find a cure for COVID-19, there had been no proven, efficacious, and widely-available treatment for the disease. With the death toll rising in various parts of the US and the world, it is imperative that we work on determining new therapeutic modalities. The present invention relates to inpatient and critical care method of treatment for COVID-19 patients as well as method of prevention of COVID-19 comprising administering at least a molecule containing Selenium (Se) for implementing it.

Substantial morbidity and mortality have been associated with the pandemic since its emergence. According to official numbers from the Centers for Disease Control and Prevention (CDC), as of Mar. 4, 2021, the total number of cases in the US is 28,514,774 with 447,569 new cases added in the previous seven days. A total death toll of 515,277 was reported by the same date. A total of 540,000 to 564,000 COVID-19 deaths is predicted by Mar. 27, 2021. Various models predict the number of hospitalizations with expected surges following national holidays.

The role of Selenium (Se) as a trace element involved in many biological processes and reactions is well established in various organisms. Particularly. Selenium is known to have anti-oxidative properties being part of various Selenoproteins including Glutathione Peroxidase (GPx), Thioredoxin Reductases (TrxR) and Selenoprotein P (SeP) which act as a scavenging system for Reactive Oxygen Species (ROS). The involvement of ROS in various pathophysiological processes is well known.

Selenium also acts as a Cytokine modulator and has been proven in multiple studies to inhibit Interleukin-6 (IL-6), Interleukin-1 (IL-1), and Tumor Necrosis Factor-alpha (TNF-a) production hence playing a major role in dampening the “Cytokine Storm” that is associated with severe inflammatory processes characteristic of Severe Inflammatory Response Syndrome (SIRS).

Selenium deficiency has been implicated in viral infections. Parts of the world where Selenium is deficient in the soil (parts of China and Europe) have been proven to be more susceptible to the emergence and evolution of multiple viral outbreaks. Se-deficient mice exhibited a significant transformation of benign viruses into highly virulent forms. An example of this is when Se-deficient mice were inoculated with a benign strain of Coxsackie virus, mutations occurred in the genome to give myocarditis with similarities to those seen in humans. Rapid change in the pathogenicity of the virus in Se-deficient hosts has been also reported for the influenza virus.

Oxidative stress plays a role in further mutating viral RNA, creating new strains. Multiple reports have shown mutations within the Severe Acute Respiratory Syndrome Coronavirus-2 (SARS CoV-2) genome since the start of the pandemic. In this regard, a study on mice that were unable to make GPx showed this enzyme essential for the avoidance of oxidative damage to the RNA-viral genome that results in the myocarditic mutations for other viruses. Therefore, increasing GPx levels via Selenium administration is protective. Evidence of the role of Selenium in combating ROS in other viral infections is also documented. Furthermore, HCV infection is an example of virus-induced generation of ROS in the liver. Moreover, plasma levels of Selenium together and erythrocyte GPx activities were significantly reduced in HCV-infected patients than in healthy controls. Multiple studies have shown the importance of Selenium in preventing the progression of HIV into AIDS through inhibition of reverse transcriptase activity in animals. Selenium has been shown to inhibit HIV replication and activation in vitro. Progressive loss of plasma Selenium has been reported to parallel the decline in CD4 T cells in HIV-1 infection. Notably, parts of Western Africa where HIV prevalence is the highest are significantly deficient in Se.

Multiple reports have emerged of the cardiotoxic effects of SARS-CoV-2, the virus causing the Coronavirus Disease 2019 (COVID-19). This has been attributed to either direct effects or as an inflammatory reaction both of which could increase ROS production. Selenium inhibits both pathways by attenuating ROS production and by inhibiting the cytokine storm.

Selenium has been shown to play a major role in the immune system. Both cell-mediated immunity and B-cell function can be impaired in Selenium deficient hosts. Selenium's ability to upregulate the expression of receptors for the growth-regulatory cytokine Interluekin-2 (IL-2) on the surface of activated lymphocytes and natural-killer cells plays an important role in clonal expansion and differentiation into cytotoxic T cells. Selenium has also been found to induce L-selectin shedding from monocytes through a metalloproteinase-dependent mechanism. This inhibition of monocyte adhesion might help explain the mechanism of action behind Se's ability to modulate inflammatory reactions.

COVID-19 is a respiratory illness that is caused by the novel SARS-CoV-2. Illness severity can widely range from mild, moderate, severe featuring pneumonia, to critical. Many experts agree that the clinical and pathological picture of critically ill COVID-19 resembles that of Acute Respiratory Distress Syndrome (ARDS) or respiratory failure requiring admission into the Intensive Care Unit (ICU) and mechanical ventilation. This is characterized by alveolar damage, surfactant abnormalities, increased alveolar capillary permeability, decreased alveolar clearance, the release of the proteinaceous fluid within the alveoli and ultimately hypoxia. A key pathway within ARDS pathophysiology is oxidative stress caused by the release of ROS. ARDS is also the leading cause of mortality in COVID-19 patients. Equally important, COVID-19 is also associated with a “Cytokine Storm” which involves massive release of inflammatory cytokines and recruitment of inflammatory cells. Inflammatory Cytokine IL-6 levels were significantly elevated in critically ill patients, which is almost 10-folds higher than those in other patients. It was therefore suggested as a therapeutic target. Early fibrosis is characteristic of Acute Lung Injury (ALI) such as that occurring in COVID-19. Cardiotoxic effects of COVID-19 have been reported which were attributed to either direct cytotoxic effects of the virus causing myocarditis and elevated cardiac enzymes or to an inflammatory reaction. One of the potential causes for nephrotoxicity is cytokine damage. Given the high morbidity and mortality of this disease, it is necessary to develop a treatment which can reduce the severity of the condition, the need for mechanical ventilation, and the associated multiple organ failures and mortality.

Selenium has been used safely for critically ill patients within recommended dosing. Multiple clinical trials have shown that it improves oxygenation, reduces illness severity, infectious complications, the need for vasoactive support and a ventilator, and 28-day mortality in the critically ill although to varying extents. The beneficial effect has been proven to be more pronounced in patients with low Selenium levels upon admission to the ICU which is the case for critically ill patients. In fact, lower Selenium levels correlated with disease severity in the critically ill. Multiple clinical studies aimed at replenishing the low Selenium levels in these patients. Sufficient evidence is available to consider initiating high-dose intravenous Selenium therapy routinely in critically ill SIRS patients, immediately on admission to the ICU.

Multiple animal models of ALI and other pulmonary illnesses including Interstitial Pulmonary Fibrosis (IPF) have established Selenium's beneficial effects. It has been particularly shown to be efficacious in mitigating the physiological and histopathological damage caused by ALI. Selenium therefore exerts various beneficial effects that are crucial in alleviating the damage that might be caused by SARS-CoV-2 in COVID-19. These include antioxidant effects through the reduction of reactive oxygen species, attenuation of lipid peroxidation, enhancement of Glutathione Peroxidase (GPx) levels, and its activation, restoration of the levels and activities of Superoxide Dismutase (SOD), Catalase and Glutathione-S-Transferase (GST) in various tissues as shown in multiple animal models. It also attenuates the histopathological patterns seen in ALIs including septal thickening and edema, hemorrhages, and inflammatory cell infiltration. Selenium also protects against cytotoxicity and lung fibrosis. Higher Selenium levels are associated with a decreased Growth Differentiation Factor-15 (GDF-15), a profibrotic cytokine, a biomarker for fibrosis, and a tool for risk stratification in critically ill ARDS patients. Viral-induced apoptosis and mucus secretion are greater in Selenium deficiency as shown by a previous study. Selenium supplementation of lung epithelial cells enhances nuclear factor E2-related factor 2 (Nrf2) activation which plays an important role in protecting against ALI/ARDS. Selenium showed similar effects on other tissues. Selenium exerts anticoagulant effects through enhancing the prostacyclin/thromboxane A2 (PGI2:TXA2) ratio hence improving the antithrombotic capacity. Microvascular damage and intrinsic fibrinogen defects due to Selenium deficiency has also been suggested. Given the thrombotic events that were reported in COVID-19 patients, Selenium could play a role in preventing these complications.

There is a lack of research studies eliciting the effects of Selenium on human Coronaviruses specifically. However, Selenium has been shown to enhance the immune response against an avian coronavirus known as Infectious Bronchitis Virus (IBV). Based on genetic analysis, IBV is classified as a Gammacoronavirus which produces a highly contagious disease in chickens. IBV produces upper respiratory tract infection, and affects the kidneys causing nephritis. Interestingly, SARS-COV-2 is also producing renal problems in severely ill patients. A recent paper from China has characterized, using computational methods, possible therapeutic molecules for SARS-CoV-2 through inhibiting the viral protein PLpro. On the list was glutathione, the production of which is enhanced by Selenium administration.

A recent study published in the American Journal of Clinical Nutrition shed the light on a significant link between regional Se status and the clinical outcomes of COVID-19 patients in China. Areas that were deficient in Se showed lower cure rates than Se-sufficient regions.

Worth noting is the low Se intake in regions of Northern Italy where COVID-19 had a devastating impact.

Pharmaconutrition with high dose Selenium particularly in the form of sodium selenite has been proven to be safe and efficacious showing reduction in illness severity, infectious complications, and is associated with decreased mortality in the ICU. Routine administration of high-dose Selenium to critically-ill SIRS has been recommended as well. High-dose Selenium in the form of sodium selenite could be administered as an IV bolus to exert antioxidant, anti-inflammatory, immune-enhancing, and antiviral properties, followed by the continuous infusion of a reduced dose for up to 14 days. Selenium exerts its effects in a dose-dependent pattern. Higher doses of Selenium have been more efficacious.

A much higher dose (2000-8000 μg) than commonly used in clinical trials has been used to treat critically ill patients suffering from SIRS with a positive and safe outcome marked particularly by a rapidly resolving ARDS. The present invention relates to a similar approach with the use of at least a molecule containing Selenium (Se) for a method of treating patients suffering from COVID-19 as well as preventing COVID-19 and its associated ARDS and SIRS, as well as associated conditions of “Cytokine Storm” and oxidative stress caused by the release of ROS.

### SUMMARY OF THE INVENTION

Generally, in one aspect of the present invention, a method for treatment of Coronavirus Disease 19 (COVID-19) and associated clinical presentations caused by novel Severe Acute Respiratory Syndrome Coronavirus-2 (SARS-CoV-2) infection, the method comprising the steps of:

administrating to a subject, a therapeutically effective amount of at least a pharmacologically acceptable molecule containing Selenium (Se) referred to as a bolus dose in an initial bolus dose administration phase;
monitoring primary and secondary outcomes with the bolus dose;
administrating to said subject, a therapeutically effective amount of at least a pharmacologically acceptable molecule containing Se referred to as a reduced, continuous dose in a successive reduced, continuous dose administration phase; and monitoring primary and secondary outcomes with the reduced, continuous dose,
wherein the therapeutically effective amount of the bolus dose is a daily dose in a range between 1000 μg per day up to 6000 μg per day, corresponding to blood Se levels in a range between 0.0125 mg/kg up to 0.075 mg/kg of bodyweight, administered daily as required,
wherein the therapeutically effective amount of the reduced, continuous dose is a daily dose in a range between 1000 μg per day up to 1600 μg per day, corresponding to blood Se levels in a range between 0.0125 mg/kg up to 0.0200 mg/kg of bodyweight, administered daily as required,
wherein the subject is a human or a subject under veterinary medicine.

In another aspect of the present invention, method for prevention or treatment of COVID-19 and associated clinical presentations caused by novel SARS-CoV-2 infection, the method comprising the steps of:

administrating to a subject, a therapeutically effective amount of at least a pharmacologically acceptable molecule containing Selenium (Se) referred to as a bolus dose in an initial bolus dose administration phase;
monitoring clinical outcomes with the bolus dose;
administrating to said subject, a therapeutically effective amount of at least a pharmacologically acceptable molecule containing Se referred to as a reduced, continuous dose in a successive reduced, continuous dose administration phase; and
monitoring clinical outcomes with the reduced, continuous dose,
wherein the therapeutically effective amount of the bolus dose is a daily dose in a range between 1000 μg per day up to 6000 μg per day, corresponding to blood Se levels in a range between 0.0125 mg/kg up to 0.075 mg/kg of bodyweight, administered daily as required,
wherein the therapeutically effective amount of the reduced, continuous dose is a daily dose in a range between 1000 μg per day up to 1600 μg per day, corresponding to blood Se levels in a range between 0.0125 mg/kg up to 0.0200 mg/kg of bodyweight, administered daily as required,
wherein the subject is monitored in terms of age, sex, ethnicity, Selenium levels, Oxygen levels, Alanine transaminase (ALT) levels, Aspartate transaminase (AST) levels, Creatinine levels, Glucose levels, Hemoglobin levels, Platelets levels, Prothrombin time (PT). C-Reactive Protein (CRP) levels, Ferritin levels. D-dimer levels, total bilirubin levels, White Blood Cell counts (WBC) with differential, Complete Blood Counts (CBC), Interleukin-1 (IL-1) levels, Interleukin-6 (IL-6) levels, Tumour Necrosis Factor-alpha (TNF-α) levels, SARS CoV-2 Polymerase Chain Reaction (PCR) test results, all other medications prescribed and consumed by said subject, adverse events, and co-morbidities, and
wherein the subject is a human or a subject under veterinary medicine.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWING

The accompanying drawings are included to provide a further understanding of the invention, and are incorporated in and constitute a part of the present invention and, together with the description, serve to explain the principle of the invention.

In the drawings,

FIG. 1 is a schematic illustration of some the key mechanisms of cellular disruption induced by the novel Severe Acute Respiratory Syndrome Coronavirus-2 (SARS-CoV-2) and their inhibition by the trace element, Selenium (Se) induced cytoprotective response involving the upregulation of various Selenoproteins including Glutathione Peroxidase (GPx). Thioredoxin Reductases (TrxR) and Selenoprotein P (SeP). It provides an illustration of the antioxidant, cytokine-modulating, antiviral, immune-enhancing, anti-apoptotic, and anticoagulant properties of Se in combating the infection caused by SARS-CoV-2.

FIG. 2 indicates is a schematic illustration of some the key mechanisms of the novel Severe Acute Respiratory Syndrome Coronavirus-2 (SARS-CoV-2) caused Coronavirus Disease 2019 (COVID-19) and its associated Acute Respiratory Distress Syndrome (ARDS) and Severe Inflammatory Response Syndrome (SIRS), and/or any state corresponding to a severe acute attack of an inflammatory pathology causing an exacerbation of cytokine release manifesting as a cytokine storm, which is further enhanced with the deficiency of the trace element Selenium (Se) mediated antioxidant, cytokine-modulating, antiviral, immune-enhancing, anti-apoptotic, and anticoagulant represented by the deficiency or absence of oxidative stress-, inflammatory stress-, and unfolded protein response (UPR)-combating Selenoproteins including Glutathione Peroxidase (GPx), Thioredoxin Reductases (TrxR) and Selenoprotein P (SeP).

FIG. 3 indicates study design and expected results in the comparative study of Selenium intravenous treatment for moderately ill, severely ill to critically ill COVID-19 patients as captured in Table 1 and Example 1 of the present invention.

FIG. 4 indicates study design and expected results in the comparative study of Selenium oral treatment (oral dosage or through feeding tube) for mildly ill to moderately ill COVID-19 patients as captured in Table 2 and Example 2 of the present invention.

FIG. 5 indicates study design and expected results in the comparative study of Selenium oral treatment available as over the counter (OTC) medication for preventive strategy for otherwise healthy individuals to prevent diseases caused by SARS-COV-2 infection and COVID-19 as captured in Table 3 and Example 3 of the present invention

### DETAILED DESCRIPTION OF THE INVENTION

The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

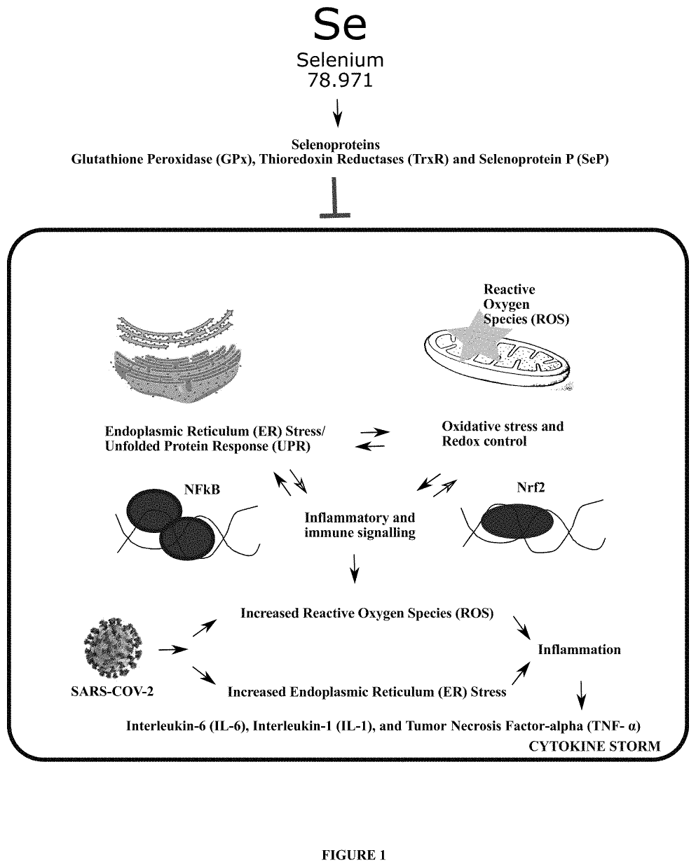

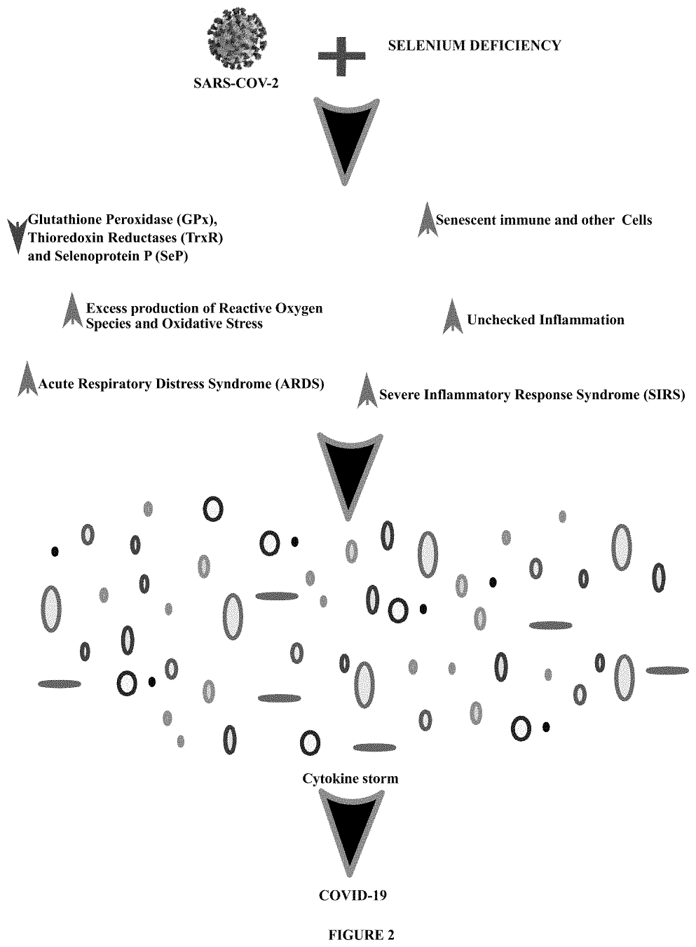

Selenium inhibits various cytotoxic pathways that are upregulated following SARS-CoV-2 infection by attenuating reactive oxygen species (ROS) production, mitochondrial oxidative stress, dampening endoplasmic reticulum (ER) stress or unfolded protein response (UPR) and by inhibiting the cytokine storm as illustrated in the schematic of FIG. 1 of the present invention. The present invention utilizes Selenium's unique properties in reducing the mortality and the incidence of visceral failures, particularly respiratory, cardiovascular, renal, coagulation failures, among others, resulting from ARDS and SIRS (including massive release of cytokines) as part of COVID-19, by using high doses of Selenium as captured in the illustration of FIG. 2 of the present invention. This is achieved by administering a drug/compound that has high dose of Selenium as initial bolus dose followed by reduced dosing in subsequent treatment. The main goal of the present invention is to use at least one Selenium-containing molecule, in an amount corresponding to a daily dose of about 1000-200 μg, and up to 6000 μg per day (corresponding to blood Se levels of 0.0125-0.075 mg/kg of bodyweight) atomic Se equivalent as a bolus dose followed by a reduced dose of 1000-1600 μg per day (corresponding to a daily dose of 0.0125-0.02 mg/kg of bodyweight) for the treatment of Coronavirus Disease 2019 (COVID-19) and any clinical presentations associated with it including Acute Respiratory Distress Syndrome (ARDS). Severe Inflammatory Response Syndrome (SIRS), and/or any state corresponding to a severe acute attack of an inflammatory pathology causing an exacerbation of cytokine release related to the novel Severe Acute Respiratory Syndrome Coronavirus-2 (SARS-CoV-2). The molecule containing Selenium maybe any pharmacologically acceptable molecule. The administration of these doses will require close monitoring and follow-up for signs of improvement or toxicity. The present invention can be implemented in human and veterinary medicine. High doses of the drug administered during the initial phase of the treatment, preferably the first one to three days comprises a sufficient amount to exert potent antioxidant, immune-enhancing, antiviral, anti-coagulant, and cytokine modulating effects, required to prevent massive inflammatory exacerbation and deterioration of clinical signs. The recommended high bolus dose and subsequent reduced doses can be adjusted according to the clinical picture and inflammatory response of the patient or animal. Indicators of this may include the need for vasoactive support (pressors), mechanical ventilation, and inflammatory state. Inflammatory state can be monitored mainly through circulating plasma levels of Interleukin-6 (IL-6). Other biomarkers include plasma levels of Interluekin-1 (IL-1), and Tumor Necrosis Factor-alpha (TNF-α).

In certain instances, as deemed necessary, as in case of a lack of significant recovery or absence of improvement, a high dose of 1000-2000 μg per day, and up to 6000 μg per day of a pharmacologically acceptable Selenium drug maybe continued further for a longer administration period (longer than the preferred three days), while maintaining close monitoring.

The efficacy and toxicity of Selenium has been shown to be dose dependent, and higher doses have been proven to be efficacious and safe. Selenium levels in the blood provide a good guide to dosing. No adverse effects have been described in clinical studies using 1000 μg per day sodium selenite constant syringe pump infusion for 14 days.

Selenium however has pro-oxidant effects and, in humans, symptoms of toxicity have been reported with whole blood Selenium levels above 1,000 μg/L.

Selenium containing molecule, or compound or drug that maybe used include any pharmacologically acceptable Selenium salt, such as a selenite or selenate of inorganic Selenium, organic Selenium, such as, selenomethionine, selenodiglutathione, selenocysteine, selenomethyl selenocysteine, dimethyl selenoxide, selenocystamine, selenated yeasts or synthetic chemicals containing one or more atoms of Selenium. The preferred molecule is sodium selenite. The Selenium-containing molecule may consist of any Selenium-containing protein that is well known in the art, preferably of a bacterial or fungal or a mammal Selenium-containing protein, including a human or a rat Selenium-containing protein.

The present invention pertains to the treatment of Coronavirus Disease 19 (COVID-19) including its clinical presentations ranging from mild, to moderate, to severe including pneumonia requiring hospitalization, to critical requiring admission to the intensive care unit and possible mechanical ventilation. This includes patients exhibiting Acute Respiratory Distress Syndrome (ARDS), Severe Inflammatory Response Syndrome (SIRS), and/or any state corresponding to a severe acute attack of an inflammatory pathology causing an exacerbation of cytokine release related to the novel Severe Acute Respiratory Syndrome (SARS-CoV-2).

The present invention pertains to a therapeutic method including two successive steps of treatment, each step of the treatment includes specific daily dosing of a Selenium-containing compound.

In accordance with one embodiment of the present invention, it discloses a method for treatment of Coronavirus Disease 19 (COVID-19) and associated clinical presentations caused by novel Severe Acute Respiratory Syndrome Coronavirus-2 (SARS-CoV-2) infection, the method comprising the steps of:

administrating to a subject, a therapeutically effective amount of at least a pharmacologically acceptable molecule containing Selenium (Se) referred to as a bolus dose in an initial bolus dose administration phase;
monitoring primary and secondary outcomes with the bolus dose;
administrating to said subject, a therapeutically effective amount of at least a pharmacologically acceptable molecule containing Se referred to as a reduced, continuous dose in a successive reduced, continuous dose administration phase; and
monitoring primary and secondary outcomes with the reduced, continuous dose,
wherein the therapeutically effective amount of the bolus dose is a daily dose in a range between 1000 μg per day up to 6000 μg per day, corresponding to blood Se levels in a range between 0.0125 mg/kg up to 0.075 mg/kg of bodyweight, administered daily as required,
wherein the therapeutically effective amount of the reduced, continuous dose is a daily dose in a range between 1000 μg per day up to 1600 μg per day, corresponding to blood Se levels in a range between 0.0125 mg/kg up to 0.0200 mg/kg of bodyweight, administered daily as required,
wherein the subject is monitored in terms of age, sex, ethnicity. Selenium levels, Oxygen levels, Alanine transaminase (ALT) levels, Aspartate transaminase (AST) levels, Creatinine levels, Glucose levels, Hemoglobin levels, Platelets levels, Prothrombin time (PT) levels, C-Reactive Protein (CRP) levels, Ferritin levels, D-dimer levels, total bilirubin levels, White Blood Cell counts (WBC) with differential levels, Complete Blood Counts (CBC) levels, Interleukin-1 (IL-1) levels, Interleukin-6 (IL-6) levels, Tumour Necrosis Factor-alpha (TNF-α) levels, SARS CoV-2 Polymerase Chain Reaction (PCR) test results, all medications prior to and during hospitalization, ventilator status and settings, as required, supplemental oxygen status, adverse events, and co-morbidities, and
wherein the subject is a human or a subject under veterinary medicine.

In another embodiment of the present invention, the method for treatment of COVID-19 and associated clinical presentations caused by novel SARS-CoV-2 infection, according to the present invention, wherein the at least a pharmacologically acceptable molecule containing Selenium (Se) is selected from the group consisting of:

a selenium hydride of the formula Sex Hy, wherein x is an integer from 1 to 10 and y has the same value than x;
a selenium salt selected from the group consisting of a fluorine salt of selenium, a chlorine salt of selenium, a bromine salt of selenium, an iodine salt of selenium, a selenium oxide, a sulphur salt of selenium, a tellurium salt of selenium a potassium salt of selenium, a sodium salt of selenium, a copper salt of selenium, a germanium salt of selenium, a barium salt of selenium, a lead salt of selenium, a zinc salt of selenium, or a nitrogen salt of selenium, particularly sodium selenite;
an inorganic selenium salt including a selenite, selenate or selenide selected from the group consisting of Antimony (III) selenide [Sb2Se3], Arsenic (III) selenide [As2Se3], Bismuth (III) selenide [Bi2Se3], Cadmium selenide [CdSe], Cobalt (II) selenide [CoSe], Mercury (II) selenide [HgSe], Selenium oxychloride, Seleninyl chloride [Cl2Ose], Selenium sulfide, Selenium disulfide [S2Se], Silver (I) selenide [Ag2Se], Indium (III) selenide [In2Se3] and Strontium selenide [SeSr]:
a selenium compound selected from the group consisting of Selenic acid [H2O4Se], Selenium dioxide [O2Se], Selenium [Se]; Selenous acid and Selenious acid [H2O3Se];
an organic selenium selected from the group of selenomethionine, selenodiglutathione, selenocysteine, selenomethyl selenocysteine, dimethyl selenoxide, and selenocystamine;
a methylated derivative of selenium;
a selenium-containing amino acid;
a selenium-containing protein selected from the group consisting of a bacterial or fungal or a mammal Selenium-containing protein:
a selenium-containing organic compound selected from the group consisting of organic compounds consisting of alkyl compounds, alicyclic compounds, cyclane compounds, terpenic compounds, aromatic compounds and heterocyclic compounds; and
selenated yeasts or synthetic chemicals containing one or more atoms of Selenium.

In another embodiment of the present invention, the method for treatment of COVID-19 and associated clinical presentations caused by novel SARS-CoV-2 infection, according to the present invention, wherein the bolus dose is administered in the initial bolus dose administration phase lasting between day 1 and day 3 of said bolus dose administration, administered daily as required.

In another embodiment of the present invention, the method for treatment of COVID-19 and associated clinical presentations caused by novel SARS-CoV-2 infection, according to the present invention, wherein the successive reduced, continuous dose administration phase lasting between day 2 and day 14 from the day of the last bolus dose administration as the two successive phases of treatment, and said reduced, continuous dose is administered daily as required, and the total administration period including the initial bolus dose administration phase and the reduced, continuous dose administration phase is 14 days.

In another embodiment of the present invention, the method for treatment of COVID-19 and associated clinical presentations caused by novel SARS-CoV-2 infection, according to the present invention, wherein the administrations of the bolus dose and the reduced, continuous dose are in the form of an injectable or pharmaceutical form.

In another embodiment of the present invention, the method for treatment of COVID-19 and associated clinical presentations caused by novel SARS-CoV-2 infection, according to the present invention, wherein the administrations of the bolus dose and the reduced, continuous dose are carried out by intravenous, subcutaneous, intramuscular, intrapentoneal, or enteral routes.

In another embodiment of the present invention, the method for treatment of COVID-19 and associated clinical presentations caused by novel SARS-CoV-2 infection, according to the present invention, wherein the administrations of the bolus dose and the reduced, continuous dose are carried out by intravenous infusion.

In another embodiment of the present invention, the method for treatment of COVID-19 and associated clinical presentations caused by novel SARS-CoV-2 infection, according to the present invention, wherein the treatment of COVID-19 includes its clinical presentations ranging from mild, to moderate, to severe including pneumonia requiring hospitalization, to critical requiring admission to the admission into the Intensive Care Unit (ICU) and mechanical ventilation.

In another embodiment of the present invention, the method for treatment of COVID-19 and associated clinical presentations caused by novel SARS-CoV-2 infection, according to the present invention, wherein the associated clinical presentations include Acute Respiratory Distress Syndrome (ARDS), Severe Inflammatory Response Syndrome (SIRS), any state corresponding to a severe acute attack of an inflammatory pathology causing an exacerbation of cytokine release and recruitment of inflammatory cells, alveolar damage, surfactant abnormalities, increased alveolar capillary permeability, decreased alveolar clearance, the release of the proteinaceous fluid within the alveoli and ultimately hypoxia, Interstitial Pulmonary Fibrosis (IPF), Acute Lung Injury (ALI), cardiotoxicity, and nephrotoxicity.

In another embodiment of the present invention, the method for treatment of COVID-19 and associated clinical presentations caused by novel SARS-CoV-2 infection, according to the present invention, wherein the administrations of the bolus dose and the reduced, continuous dose are carried out in addition to the Standard of Care (SOC) treatment.

In another embodiment of the present invention, the method for treatment of COVID-19 and associated clinical presentations caused by novel SARS-CoV-2 infection, according to the present invention, wherein the SOC treatment is selected from the group consisting of steroids including Dexamethasone, antibiotics including Azithromycin, and Ceftriaxone, anti-viral including Remdesivir, and Convalescent Plasma or a combination thereof.

In another embodiment of the present invention, the method for treatment of COVID-19 and associated clinical presentations caused by novel SARS-CoV-2 infection, according to the present invention, wherein the monitoring primary and secondary outcomes with the bolus dose in the initial bolus dose administration phase and the monitoring primary and secondary outcomes with the reduced, continuous dose in the successive reduced, continuous dose administration phase is carried out for a total period of 29 days or until discharge or death, starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, are measured daily for a total period of 29 days or until discharge or death, wherein the CRP levels, IL-1 levels, IL-6 levels, and TNF-α are measured on day 1, day 3, day 5, day 7, day 10, day 14, day 21, and day 29, and wherein the Selenium levels are measured on day 1 and day 29, starting from day 1 of the initial bolus dose administration phase.

In another embodiment of the present invention, the method for treatment of COVID-19 and associated clinical presentations caused by novel SARS-CoV-2 infection, according to the present invention, wherein the monitoring primary outcomes comprises calculation of the rate of hospital discharges or deaths, wherein a subject is followed until hospital discharge, or death from the date of admission.

In another embodiment of the present invention, the method for treatment of COVID-19 and associated clinical presentations caused by novel SARS-CoV-2 infection, according to the present invention, wherein 1. The monitoring secondary outcomes comprises calculations of:

a. change from baseline in alanine transaminase (ALT) measured in a time frame of day 1 through day 29;
b. change from baseline in aspartate transaminase (AST) measured in a time frame of day 1 through day 29.
c. change from baseline in creatinine measured in a time frame of day 1 through day 29;
d. change from baseline in glucose measured in a time frame of day 1 through day 29;
e. change from baseline in hemoglobin measured in a time frame of day 1 through day 29;
f. change from baseline in platelets measured in a time frame of day 1 through day 29;
g. change from baseline in prothrombin time (PT) measured in a time frame of day 1 through day 29;
h. change from baseline in total bilirubin measured in a time frame of day 1 through day 29;
i. change from baseline in white blood cell count (WBC) with differential measured in a time frame of day 1 through day 29;
j. change in National Early Warning Score (NEWS) from baseline measured in a time frame of day 1 through day 29;
k. clinical status using ordinal scale measured in a time frame of day 1 through day 29;
l. cumulative incidence of serious adverse events (SAEs) measured in a time frame of day 1 through day 29;
m. discontinuation or temporary suspension of investigational therapeutics measured in a time frame of day 1 through day 14,
n. duration of hospitalization measured in a time frame of day 1 through day 29;
o. duration of new non-invasive ventilation or high flow oxygen use measured in a time frame of day 1 through day 29;
p. duration of new oxygen use measured in a time frame of day 1 through day 29:
q. duration of new ventilator use measured in a time frame of day 1 through day 29;
r. incidence of new non-invasive ventilation or high flow oxygen use measured in a time frame of day 1 through day 29;
s. incidence of new oxygen use measured in a time frame of day 1 through day 29;
t. incidence of new ventilator use measured in a time frame of day 1 through day 29;
u. mean change in the ordinal scale measured in a time frame of day 1 through day 29; and
v. time to an improvement of one category using an ordinal scale measured in a time frame of day 1 through day 29,
wherein day 1 is the day 1 of the initial bolus dose administration phase,
wherein the NEWS has demonstrated an ability to discriminate patients at risk of poor outcomes and it is based on 7 clinical parameters consisting of respiration rate, oxygen saturation, any supplemental oxygen, temperature, systolic blood pressure, heart rate, level of consciousness,
wherein the ordinal scale is an assessment of the clinical status at the first assessment of a given study day and is provided as a scale ranging from: i) death; ii) hospitalized, on invasive mechanical ventilation; iii) hospitalized, on non-invasive ventilation or high flow oxygen devices; iv) hospitalized, requiring supplemental oxygen; v) hospitalized, not requiring supplemental oxygen—requiring ongoing medical care (COVID-19 related or otherwise); vi) hospitalized, not requiring supplemental oxygen—no longer requires ongoing medical care; vii) not hospitalized, limitation on activities and/or requiring home oxygen; and viii) not hospitalized, no limitations on activities, and
wherein the SAEs are defined in terms of an adverse event (AE) or suspected adverse reaction when considered serious in the view of either the investigator, it results in death, a life-threatening AE, inpatient hospitalization or prolongation of existing hospitalization, a persistent or significant incapacity or substantial disruption of the ability to conduct normal life functions.

In another embodiment of the present invention, the method for treatment of COVID-19 and associated clinical presentations caused by novel SARS-CoV-2 infection, according to the present invention, wherein the bolus dose in an initial bolus dose administration phase, or the reduced, continuous dose in a successive reduced, continuous dose administration phase, or both the bolus dose in an initial bolus dose administration phase, the reduced, continuous dose in a successive reduced, continuous dose administration phase, is in combination with a therapeutically effective quantity of at least a non-selenium compound, wherein the non-selenium compound is capable of inhibiting oxidative metabolism or acting against the consequences of oxidative stress or inhibiting the inflammatory reaction or exhibiting antiviral properties or exhibiting antiapoptotic properties or a combination thereof.

In one embodiment of the present invention, a method for prevention or treatment of COVID-19 and associated clinical presentations caused by novel SARS-CoV-2 infection, the method comprising the steps of:

administrating to a subject, a therapeutically effective amount of at least a pharmacologically acceptable molecule containing Selenium (Se) referred to as a bolus dose in an initial bolus dose administration phase:
monitoring clinical outcomes with the bolus dose;
administrating to said subject, a therapeutically effective amount of at least a pharmacologically acceptable molecule containing Se referred to as a reduced, continuous dose in a successive reduced, continuous dose administration phase; and
monitoring clinical outcomes with the reduced, continuous dose,
wherein the therapeutically effective amount of the bolus dose is a daily dose in a range between 1000 μg per day up to 6000 μg per day, corresponding to blood Se levels in a range between 0.0125 mg/kg up to 0.075 mg/kg of bodyweight, administered daily as required,
wherein the therapeutically effective amount of the reduced, continuous dose is a daily dose in a range between 1000 μg per day up to 1600 μg per day, corresponding to blood Se levels in a range between 0.0125 mg/kg up to 0.0200 mg/kg of bodyweight, administered daily as required,
wherein the subject is monitored in terms of age, sex, ethnicity. Selenium levels, Oxygen levels, Alanine transaminase (ALT) levels, Aspartate transaminase (AST) levels, Creatinine levels, Glucose levels, Hemoglobin levels, Platelets levels, Prothrombin time (PT), C-Reactive Protein (CRP) levels, Ferritin levels, D-dimer levels, total bilirubin levels, White Blood Cell counts (WBC) with differential, Complete Blood Counts (CBC), Interleukin-1 (IL-1) levels, Interleukin-6 (IL-6) levels, Tumour Necrosis Factor-alpha (TNF-α) levels, SARS CoV-2 Polymerase Chain Reaction (PCR) test results, all other medications prescribed and consumed by said subject, adverse events, and co-morbidities, and
wherein the subject is a human or a subject under veterinary medicine.

In another embodiment of the present invention, the method for prevention or treatment of COVID-19 and associated clinical presentations caused by novel SARS-CoV-2 infection, according to the present invention, wherein the administrations of the bolus dose and the reduced, continuous dose are carried out by oral route or through a feeding tube, wherein the monitoring primary and secondary outcomes with the bolus dose in the initial bolus dose administration phase and the monitoring primary and secondary outcomes with the reduced, continuous dose in the successive reduced, continuous dose administration phase is carried out for a total period of 29 days or until discharge or death, starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, CRP levels, IL-1 levels, IL-6 levels, and TNF-α are measured on day 1, day 14, and day 29, and wherein the Selenium levels are measured on day 1 and day 29, starting from day 1 of the initial bolus dose administration phase.

In another embodiment of the present invention, the method for prevention or treatment of COVID-19 and associated clinical presentations caused by novel SARS-CoV-2 infection, according to the present invention, wherein the at least a pharmacologically acceptable molecule containing Selenium (Se) is selected from the group consisting of:

a selenium hydride of the formula Sex Hy, wherein x is an integer from 1 to 10 and y has the same value than x;
a selenium salt selected from the group consisting of a fluorine salt of selenium, a chlorine salt of selenium, a bromine salt of selenium, an iodine salt of selenium, a selenium oxide, a sulphur salt of selenium, a tellurium salt of selenium a potassium salt of selenium, a sodium salt of selenium, a copper salt of selenium, a germanium salt of selenium, a barium salt of selenium, a lead salt of selenium, a zinc salt of selenium, or a nitrogen salt of selenium;
an inorganic selenium salt including a selenite, selenate or selenide selected from the group consisting of Antimony (III) selenide [Sb2Se3], Arsenic (III) selenide [As2Se3], Bismuth (III) selenide [Bi2Se3], Cadmium selenide [CdSe], Cobalt (II) selenide [CoSe], Mercury (II) selenide [HgSe], Selenium oxychloride, Seleninyl chloride [Cl2Ose], Selenium sulfide. Selenium disulfide [S2Se], Silver (I) selenide [Ag2Se], Indium (III) selenide [In2Se3] and Strontium selenide [SeSr];
a selenium compound selected from the group consisting of Selenic acid [H2O4Se], Selenium dioxide [O2Se], Selenium [Se]; Selenous acid and Selenious acid [H2O3Se]:
an organic selenium selected from the group of selenomethionine, selenodiglutathione, selenocysteine, selenomethyl selenocysteine, dimethyl selenoxide, and selenocystamine;
a methylated derivative of selenium:
a selenium-containing amino acid;
a selenium-containing protein selected from the group consisting of a bacterial or fungal or a mammal Selenium-containing protein:
a selenium-containing organic compound selected from the group consisting of organic compounds consisting of alkyl compounds, alicyclic compounds, cyclane compounds, terpenic compounds, aromatic compounds and heterocyclic compounds; and
selenated yeasts or synthetic chemicals containing one or more atoms of Selenium.

In another embodiment of the present invention, the method for prevention or treatment of COVID-19 and associated clinical presentations caused by novel SARS-CoV-2 infection, according to the present invention, wherein the monitoring the clinical outcomes comprises calculations of:

a. change from baseline in alanine transaminase (ALT) measured in a time frame of day 1 through day 29;
b. change from baseline in aspartate transaminase (AST) measured in a time frame of day 1 through day 29;
c. change from baseline in creatinine measured in a time frame of day 1 through day 29;
d. change from baseline in glucose measured in a time frame of day 1 through day 29;
e. change from baseline in hemoglobin measured in a time frame of day 1 through day 29;
f. change from baseline in platelets measured in a time frame of day 1 through day 29;
g. change from baseline in prothrombin time (PT) measured in a time frame of day 1 through day 29;
h. change from baseline in total bilirubin measured in a time frame of day 1 through day 29;
i. change from baseline in white blood cell count (WBC) with differential measured in a time frame of day 1 through day 29;
j. change in National Early Warning Score (NEWS) from baseline measured in a time frame of day 1 through day 29;
k. clinical status using ordinal scale measured in a time frame of day 1 through day 29;
l. cumulative incidence of serious adverse events (SAEs) measured in a time frame of day 1 through day 29;
m. discontinuation or temporary suspension of investigational therapeutics measured in a time frame of day 1 through day 14;
n. duration of hospitalization measured in a time frame of day 1 through day 29;
o. duration of new non-invasive ventilation or high flow oxygen use measured in a time frame of day 1 through day 29;
p. duration of new oxygen use measured in a time frame of day 1 through day 29:
q. duration of new ventilator use measured in a time frame of day 1 through day 29;
r. incidence of new non-invasive ventilation or high flow oxygen use measured in a time frame of day 1 through day 29;
s. incidence of new oxygen use measured in a time frame of day 1 through day 29;
t. incidence of new ventilator use measured in a time frame of day 1 through day 29;
u. mean change in the ordinal scale measured in a time frame of day 1 through day 29; and
v. time to an improvement of one category using an ordinal scale measured in a time frame of day 1 through day 29,
wherein day 1 is the day 1 of the initial bolus dose administration phase,
wherein the NEWS has demonstrated an ability to discriminate patients at risk of poor outcomes and it is based on 7 clinical parameters consisting of respiration rate, oxygen saturation, any supplemental oxygen, temperature, systolic blood pressure, heart rate, level of consciousness,
wherein the ordinal scale is an assessment of the clinical status at the first assessment of a given study day and is provided as a scale ranging from: i) death; ii) hospitalized, on invasive mechanical ventilation; iii) hospitalized, on non-invasive ventilation or high flow oxygen devices; iv) hospitalized, requiring supplemental oxygen; v) hospitalized, not requiring supplemental oxygen—requiring ongoing medical care (COVID-19 related or otherwise); vi) hospitalized, not requiring supplemental oxygen—no longer requires ongoing medical care; vii) not hospitalized, limitation on activities and/or requiring home oxygen; and viii) not hospitalized, no limitations on activities, and
wherein the SAEs are defined in terms of an adverse event (AE) or suspected adverse reaction when considered serious in the view of either the investigator, it results in death, a life-threatening AE, inpatient hospitalization or prolongation of existing hospitalization, a persistent or significant incapacity or substantial disruption of the ability to conduct normal life functions.

In another embodiment of the present invention, the method for prevention or treatment of COVID-19 and associated clinical presentations caused by novel SARS-CoV-2 infection, according to the present invention, wherein the bolus dose in an initial bolus dose administration phase, or the reduced, continuous dose in a successive reduced, continuous dose administration phase, or both the bolus dose in an initial bolus dose administration phase, the reduced, continuous dose in a successive reduced, continuous dose administration phase, is in combination with a therapeutically effective quantity of at least a non-selenium compound, wherein the non-selenium compound is capable of inhibiting oxidative metabolism or acting against the consequences of oxidative stress or inhibiting the inflammatory reaction or exhibiting antiviral properties or exhibiting antiapoptotic properties or a combination thereof.

According to the treatment method mentioned above, a Selenium-containing molecule that can be used can be any of the molecules that are described in the present specification.

The present invention has the further object of the use of at least one molecule of Selenium such as defined above, in combination with a therapeutically effective quantity of at least one non-selenium compound inhibiting oxidative metabolism or acting against the consequences of oxidative stress or inhibiting the inflammatory reaction or exhibiting antiviral properties or exhibiting antiapoptotic properties. Various compounds inhibiting oxidative metabolism or exhibiting antioxidant properties may be used, in a drug according to the present invention, in combination with at least one molecule containing Selenium. A drug according to the present invention comprises, in combination with the molecule or molecules containing Selenium, Vitamin E, optionally combined with vitamin C, a or precursor of Glutathione, such as N-acetylcysteine.

The drug is preferably in the form of an injectable or perfusable pharmaceutical form or for enteral administration. Since perfusion is the commonly used route in the intensive care setting, it is the preferred method of administration. It may however be in any form which allows the administration of the molecule or molecules containing Selenium and the effective treatment of COVID-19 and associated presentations or sequelae. This drug may be administered by the parenteral route, preferably by intravenous, also by subcutaneous, intramuscular, and also by intraperitoneal, enteral or oral routes.

The drug is intended to be curative, but maybe administered prophylactically if deemed necessary particularly whenever exposure to SARS-CoV-2 has occurred or expected. Dosing forms and quantities should be modified accordingly to prevent toxicities.

Although the invention has been explained in relation to its preferred embodiment, it is to be understood that many other possible modifications and variations can be made without departing from the spirit and scope of the invention.

The invention will be further explained by the following Examples, which are intended to purely exemplary of the invention, and should not be considered as limiting the invention in any way.

### EXAMPLES

### Example 1—Selenium Intravenous Treatment for Moderately Ill, Severely Ill to Critically Ill COVID-19 Patients

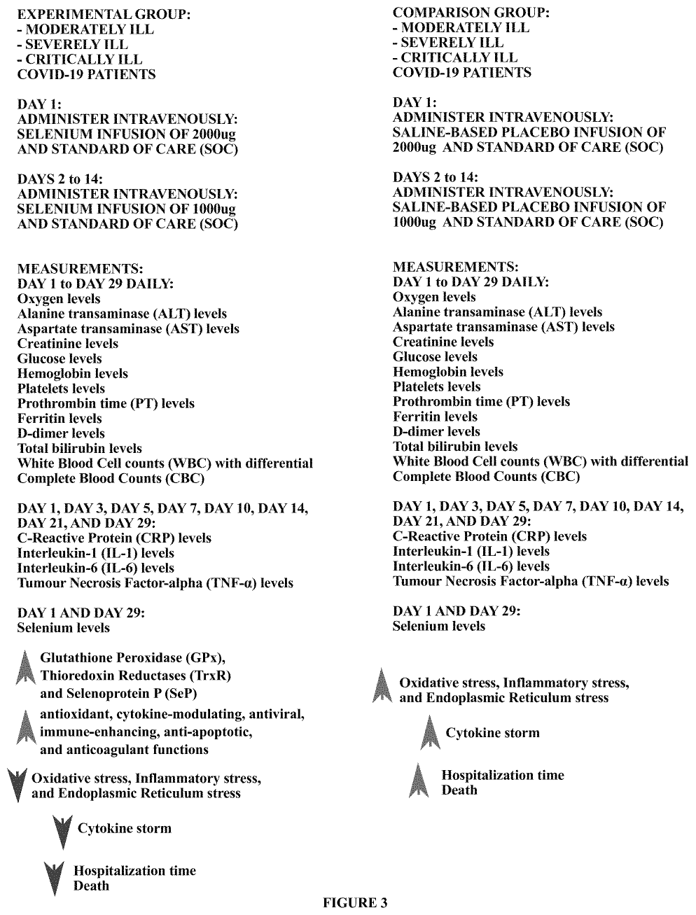

An interventional randomized clinical trial study is designed for testing the safety and effectiveness in a method of treatment for moderately ill, severely ill to critically ill COVID-19 patients with the use of Selenium and at least a pharmacologically acceptable molecule containing Selenium (Se), which in this example is Selenious Acid (from American Regent) and/or Sodium Selenite, particularly, Selenase: Sodium Selenite Pentahydrate. In the trial, eligible patients would be allocated to a 2:1 selenium: no selenium ratio through electronic randomization performed by the research team on the day of admission to take selenium or not. Patients were followed-up until admission to ICU, hospital discharge, or death as shown in Table 1 and in FIG. 3 showing the experimental design and expected results to show safety and efficacy of the proposed treatment.

As shown in the Table 1 below the experimental design envisioned for the trial is as follows:

TABLE 1
Experimental design for the experimetal group of patients to be administeted with
an intervention/treatment comprising a pharmacologically acceptable molecule
containing Selenium (Se) along with Standard of Care (SOC) and the comparison
group of patients who would be administered with SOC along with a placebo with
regards to the pharmacologically acceptable molecule containing Se as described below.

| Group | Intervention/treatment |
|---|---|
| (v) Experimental Group: | Drug: Selenium as Selenious Acid |
| to be administered | (AMERICAN REGENT ™) |
| with Selenious Add + | Administered as an intravenous infusion. |
| Standard of Care | |
| (SOC) | |
| Participants who are moderately- | Drug: Standard of Care (SOC) |
| ill, severely-ill, or critically ill | Standard of Care Treatment for |
| will receive continued standard of | COVID-19 Infection. |
| care therapy together with | |
| Selenium infusion of 2000 μg on | |
| day 1 as a loading dose infusion, | |
| followed by a continuous infusion | |
| of Selenium at a maintenance | |
| dose of 1000 μg daily on days 2- | |
| 14. | |
| (ii) Comparison Group: to be | Drug: Standard of Care (SOC) + Saline-based |
| administered Standard of Care | Placebo Standard of Care Treatment for COVID- |
| (SOC) + Placebo | 19 Infection. |
| Participants will receive a saline- | |
| based placebo infusion of 2000 μg | |
| on day 1 as a loading dose, | |
| followed by continuous infusion | |
| of a Saline-based placebo at a | |
| maintenance dose of 1000 μg | |
| daily on days 2-14. SOC is to be | |
| determined according to patients' | |
| clinical picture and may include | |
| Dexamethasone, Azithromycin, | |
| Ceftriaxone, Remdesivir, | |
| Convalescent Plasma. | |

The study is planned for a duration of four months where, the first three months would include patient enrollment and data collection, one month for data analysis and publishing. Once a COVID-19 patient who meets the inclusion criteria is selected, and their consent is obtained, they will be randomly assigned to a study group and will receive the treatment regimen accordingly. They will be monitored over the following 29 days or until discharge or death.

The study is designed keeping in mind the various aspects for selection of the study subjects as follows:

Study population: Hospitalized COVID-19 patients in inpatient wards, intermediate care units, and intensive care units.

Recruitment: Patients with COVID-19 who exhibit the signs and symptoms of moderate or severe infection or are critically ill will be recruited by the principal investigator, co-investigators, and delegated clinical staff at CHRISTUS Good Shepherd Medical Center. This study is completely voluntary and subjects are free to withdraw from the research at any time.

The maximum number of study subjects: 100 patients. This number includes anticipated screening and consent failures.

Inclusion Criteria:

a) Willing and able to provide written informed consent, or with a legal representative who can provide informed consent, or enrolled under International Conference on Harmonization (ICH) E6(R2) 4.8.15 emergency use provisions as deemed necessary by the investigator (age ≥18) prior to performing study procedures
b) Aged ≥18 years
c) Severe Acute Respiratory Syndrome Coronavirus (SARS-CoV)-2 infection confirmed by polymerase chain reaction (PCR) test ≤4 days before randomization
d) Currently hospitalized
e) Peripheral capillary oxygen saturation (SpO2)≤94% or requiring supplemental oxygen at screening

Exclusion Criteria

a) Participation in any other clinical trial of an experimental treatment for COVID-19
b) Evidence of multiorgan failure
c) Mechanically ventilated for >5 days
d) Alanine Aminotransferase (ALT) or aspartate aminotransferase (AST)>5×upper limit of normal (ULN)
e) Creatinine clearance <50 mL/min

It is expected that based on clinical conditions, other protocol defined Inclusion/Exclusion criteria may apply. Patient consent would be needed for participation in this study. Spanish-language consents will be made available.

For sample size calculation a Power analysis for a paired sample t-test was conducted in G*Power to determine a sufficient sample size using an alpha of 0.05, a power of 0.80, a medium effect size (dz=0.5), and two tails (Faul et al., 2013). Based on the aforementioned assumptions, a sample size of 34 participants in the experimental arm and 34 participants in the standard of care arm are needed to determine that selenium acts as a therapeutic agent for moderate, severe, and critical COVID-19 infections. [Faul, F., Erdfelder, E., Buchner, A., & Lang, A.-G. (2013). G*Power Version 3.1.9.7 [computer software]. Uiversitat Kiel, Germany. Retrieved from http://www.psychologie.hhu.de/arbeitsgruppen/allgemeine-psychologie-und-arbeitspsychologie/gpower.html/]

The data that is going to be collected includes the following characteristics per patient.

### 1. Medical Record Number

### 2. Age

### 3. Sex

### 4. Ethnicity

### 5. Date of Birth

6. Date of admission
7. Date of discharge, or death (if applicable)
8. Selenium levels
9. Oxygen saturation
10. Alanine transaminase (ALT) levels
it. Aspartate transaminase (AST) levels
12. Creatinine levels
13. Glucose levels
14. Hemoglobin levels
15. Platelets levels
16. Prothrombin time (PT)
17. C-Reactive Protein (CRP) levels
18. Ferritin levels
19. D-dimer levels
20. Total bilirubin levels
21. White Blood Cell counts (WBC) with differential

### 22. Complete Blood Counts (CBC)

23. Interleukin-1 levels
24. Interleukin-6 levels
25. TNF-alpha levels
26. SARS CoV-2 Polymerase Chain Reaction (PCR) test results
27. All medications prior to and during hospitalization
28. Ventilator status and settings, if applicable
29. Supplemental oxygen status
30. Adverse events

### 31. Co-morbidities

The aforementioned data will be collected via Chart review. The tool used to gather the data will primarily be an Excel spreadsheet. Principal investigator and co-investigators will be responsible for data collection. Only the medical records of research subjects who have signed consent documents will be viewed for the research study. Any data that will be analyzed for bioinformatics and biostatistics will be de-identified.

The collected data would then be analyzed by a biostatistician, the principal investigator, and co-investigators. Computer packages that would be used in the data entry and analysis may include MS Excel, SPSS, and GraphPad Prism. Other packages might be used and will be promptly reported. Types of statistical tests to be used for analysis will include t-test for qualitative variables, χ2 tests and Fisher exact tests, univariate and multivariate logistic regressions.

Medical records of research subjects who have signed consent documents will be viewed for the research study. Clinical and demographic data including name, age, gender, ethnicity, and past and present clinical history and therapy, and clinical laboratory tests will be recorded. Any data that will be analyzed for bioinformatics and biostatistics will be de-identified.

The trial is designed as a double-blinded study. Neither the participants nor investigators will be aware of participants assigned to any of the study groups. All participants will have an equal chance of being selected to the intervention arm. All patients will receive the standard of care treatment, hence, none of the subjects will be deprived of any available optimal treatment.

Risks associated with the study intervention include failure to improve or worsening of patients' conditions, and adverse events associated with selenium. Side effects of selenium are minimal. Possible side effects include muscle tenderness, tremors, lightheadedness, and facial flushing, and bleeding tendency.

The dose proposed in this clinical trial is much lower than known toxic doses. The safety of selenium at the proposed dosing has been proven in various studies (refer to reference number 30, 31, 33, 36, and 65). Nevertheless, acute poisoning typically involves a single dose that rapidly produces symptoms within minutes to hours. Selenium toxicity is determined based on the patient's clinical picture and not by laboratory values. Signs of acute toxicity include hypotension and tachycardia. Electrocardiogram abnormalities include T-wave flattening and inversion, and a prolonged QT interval. Nausea, vomiting, diarrhea, and abdominal pain are often present, and pulmonary edema can be a serious complication. Neurological symptoms include tremors, muscle spasms, irritability, confusion, delirium, and coma. Death is preceded by refractory hypotension from peripheral vasodilatation and direct myocardial depression (refer to reference number 68, and 69).

The various parameters as mentioned above shall be measured daily from day 1 to day 29 or until discharge or death, starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, are measured daily for a total period of 29 days or until discharge or death, wherein the CRP levels, IL-1 levels, IL-6 levels, and TNF-α are measured on day 1, day 3, day 5, day 7, day 10, day 14, day 21, and day 29, and wherein the Selenium levels are measured on day 1 and day 29, starting from day 1 of the initial bolus dose administration phase.

In this example as discussed in the experimental protocol presented above, in order to evaluate the safety and effectiveness of the use of Selenium as a treatment option for novel coronavirus SARS-CoV-2 infection, patients with COVID-19 who exhibit the signs and symptoms of moderate or severe infection or are critically ill will receive Selenium infusion for 14 days. This experiment is designed to demonstrate that Selenium treatment would decrease the death rates and increase the rate of hospital discharges among hospitalized COVID-19 patients.

In order to establish that, help would be taken by measuring and analyzing both primary and secondary clinical outcome measurements. The primary outcome to be measured is the rate of hospital discharges or deaths and looks at patients to be followed from admission until hospital discharge, or death.

The secondary outcomes to be measured would evaluate the following parameters:

1. Change from baseline in alanine transaminase (ALT) [Time Frame: Day 1 through Day 29]
2. Change from baseline in aspartate transaminase (AST) [Time Frame: Day 1 through Day 29]
3. Change from baseline in creatinine [Time Frame: Day 1 through Day 29]
4. Change from baseline in glucose [Time Frame: Day 1 through Day 29]
5. Change from baseline in hemoglobin [Time Frame: Day 1 through Day 29]
6. Change from baseline in platelets [Time Frame: Day 1 through Day 29]
7. Change from baseline in prothrombin time (PT) [Time Frame: Day 1 through Day 29]
8. Change from baseline in total bilirubin [Time Frame: Day 1 through Day 29]
9. Change from baseline in white blood cell count (WBC) with differential [Time Frame: Day 1 through Day 29]
10. Change in National Early Warning Score (NEWS) from baseline [Time Frame: Day 1 through Day 29]
The NEW score has demonstrated an ability to discriminate patients at risk of poor outcomes. This score is based on 7 clinical parameters (respiration rate, oxygen saturation, any supplemental oxygen, temperature, systolic blood pressure, heart rate, level of consciousness).
The NEW Score is being used as an efficacy measure.
11. Clinical status using ordinal scale [Time Frame: Day 1 through Day 29]
The ordinal scale is an assessment of the clinical status at the first assessment of a given study day. The scale is as follows: 1) Death; 2) Hospitalized, on invasive mechanical ventilation; 3) Hospitalized, on noninvasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.
12. Cumulative incidence of serious adverse events (SAEs) [Time Frame: Day 1 through Day 29]
An SAE is defined as an AE or suspected adverse reaction is considered serious if, in the view of either the investigator, it results in death, a life-threatening AE, inpatient hospitalization or prolongation of existing hospitalization, a persistent or significant incapacity or substantial disruption of the ability to conduct normal life functions.
13. Discontinuation or temporary suspension of investigational therapeutics [Time Frame: Day 1 through Day 14]
For any reason.
14. Duration of hospitalization [Time Frame: Day 1 through Day 29]
Measured in days.
15. Duration of new non-invasive ventilation or high flow oxygen use [Time Frame: Day 1 through Day 29]
Measured in days.
16. Duration of new oxygen use [Time Frame: Day 1 through Day 29]
Measured in days.
17. Duration of new ventilator use [Time Frame: Day 1 through Day 29]
Measured in days.
18. Incidence of new non-invasive ventilation or high flow oxygen use [Time Frame: Day 1 through Day 29]
19. Incidence of new oxygen use [Time Frame: Day 1 through Day 29]
20. Incidence of new ventilator use [Time Frame: Day 1 through Day 29]
21. Mean change in the ordinal scale [Time Frame: Day 1 through Day 29]
The ordinal scale is an assessment of the clinical status at the first assessment of a given study day. The scale is as follows: 1) Death; 2) Hospitalized, on invasive mechanical ventilation; 3) Hospitalized, on noninvasive ventilation or high flow oxygen devices; 4) Hospitalized, requiring supplemental oxygen: 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.
22. Time to an improvement of one category using an ordinal scale [Time Frame. Day 1 through Day 29]
The ordinal scale is an assessment of the clinical status at the first assessment of a given study day. The scale is as follows: 1) Death: 2) Hospitalized, on invasive mechanical ventilation: 3) Hospitalized, on noninvasive ventilation or high flow oxygen devices; 4) Hospitalized, requiring supplemental oxygen: 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

### Example 2—Selenium Oral Treatment for Mildly Ill, to Moderately Ill COVID-19 Patients

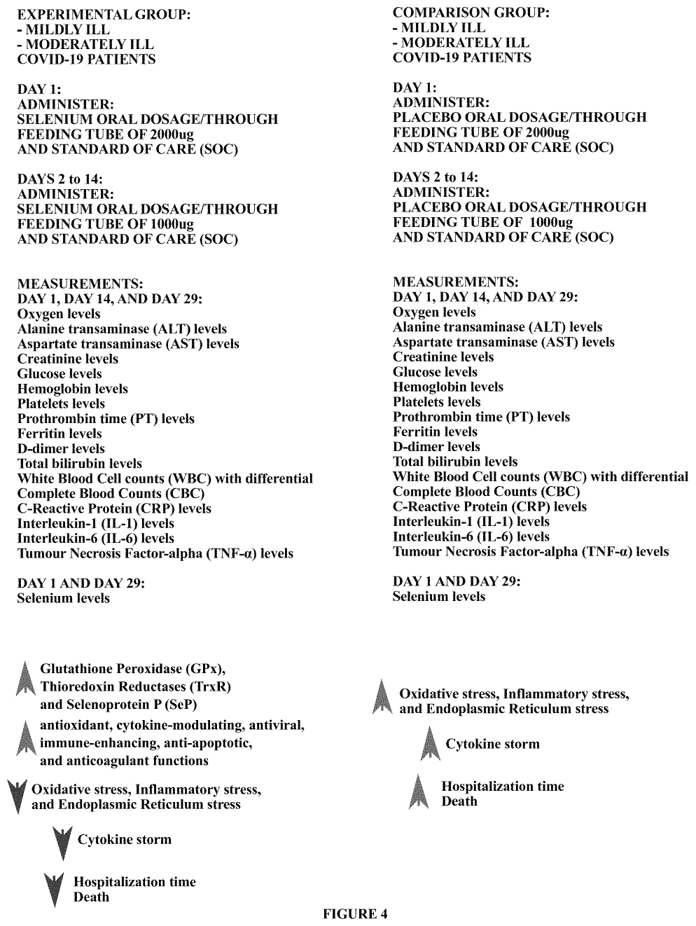

Similar to the experimental design provided hereinabove, another proposed clinical trial entails administering to mildly ill, moderately ill, severely ill to critically ill COVID-19 patients, an oral dosage regimen of Selenium treatment as provided in the experimental set-up of Table 2 hereinbelow and in FIG. 4 showing the experimental design and expected results to show safety and efficacy of the proposed treatment.

As shown in the Table 2 below the experimental design envisioned for the trial is as follows:

TABLE 2
Experimental design for the experimetal group of patients to be administeted
with an intervention/treatment comprising a pharmacologically acceptable molecule
containing Selenium (Se) along with Standard of Care (SOC) and the comparison group
of patients who would be administered with SOC along with a placebo with regards to
the pharmacologically acceptable molecule containing Se as described below.

| Group | Intervention/treatment |
|---|---|
| (v) Experimental Group: | Drug: Selenium as Selenious Acid |
| to be administered | (AMERICAN REGENT ™) |
| with Selenious Acid + | Administered as an oral dosage or |
| Standard of Care | through a feeding tube. |
| (SOC) | |
| Participants who are mildly-ill, | Drug: Standard of Care (SOC) |
| moderately-ill, severely-ill, or | Standard of Care Treatment for mild |
| critically ill will receive | to moderate COVID-19 Intfection. |
| continued standard of care | |
| therapy together with Selenium | |
| oral dosage of 2000 μg on day 1 | |
| as a loading dose, followed by a | |
| continuous oral dosage of | |
| Selenium at a maintenance dose | |
| of 1000 μg daily on days 2-14. | |
| (ii) Comparison Group: to be | Drug: Standard of Care (SOC) + Saline-based |
| administered Standard of Care | Placebo Standard of Care Treatment for mild to |
| (SOC) + Placebo | moderate COVID-19 Infection. |
| Participants will receive a saline- | |
| based placebo oral dosage of | |
| 2000 μg on day I as a loading | |
| dose, followed by continuous oral | |
| dosage of a Saline-based placebo | |
| at a maintenance dose of 1000 μg | |
| daily on days 2-14. SOC is to be | |
| determined according to patients' | |
| clinical picture and may include | |
| Dexamethasone, Azithromycin, | |
| Ceftriaxone, Remdesivir, | |
| Convalescent Plasma. | |

The study is planned for a duration of four months where, the first three months would include patient enrollment and data collection, one month for data analysis and publishing. Once a COVID-19 patient who meets the inclusion criteria is selected, and their consent is obtained, they will be randomly assigned to a study group and will receive the treatment regimen accordingly. They will be monitored over the following 29 days or until discharge or death.

The study is designed keeping in mind the various aspects for selection of the study subjects as follows:

Study population: Outpatient and/or hospitalized COVID-19 patients in inpatient wards, who have mild to moderate COVID-19.

Recruitment: Patients with COVID-19 who exhibit the signs and symptoms of moderate or severe infection or are critically ill will be recruited by the principal investigator, co-investigators, and delegated clinical staff at CHRISTUS Good Shepherd Medical Center. This study is completely voluntary and subjects are free to withdraw from the research at any time.

The maximum number of study subjects: 100 patients. This number includes anticipated screening and consent failures.

Inclusion Criteria:

a) Willing and able to provide written informed consent, or with a legal representative who can provide informed consent, or enrolled under International Conference on Harmonization (ICH) E6(R2) 4.8.15 emergency use provisions as deemed necessary by the investigator (age ≥18) prior to performing study procedures
b) Aged ≥18 years
c) Severe Acute Respiratory Syndrome Coronavirus (SARS-CoV)-2 infection confirmed by polymerase chain reaction (PCR) test ≤4 days before randomization

Exclusion Criteria

a) Participation in any other clinical trial of an experimental treatment for COVID-19
b) Evidence of multiorgan failure
c) Mechanically ventilated for >5 days
d) Alanine Aminotransferase (ALT) or aspartate aminotransferase (AST) >5×upper limit of normal (ULN)
e) Creatinine clearance <50 mL/min

It is expected that based on clinical conditions, other protocol defined Inclusion/Exclusion criteria may apply. Patient consent would be needed for participation in this study. Spanish-language consents will be made available.

For sample size calculation a Power analysis for a paired sample t-test was conducted in G*Power to determine a sufficient sample size using an alpha of 0.05, a power of 0.80, a medium effect size (dz=0.5), and two tails (Faul et al., 2013). Based on the aforementioned assumptions, a sample size of 34 participants in the experimental arm and 34 participants in the standard of care arm are needed to determine that selenium acts as a therapeutic agent for moderate, severe, and critical COVID-19 infections. [Faul, F., Erdfelder, E., Buchner, A., & Lang, A.-G. (2013). G*Power Version 3.1.9.7 [computer software]. Uiversitat Kiel. Germany. Retrieved from http://www.psychologie.hhu.de/arbeitsgruppen/allgemeine-psychologie-und-arbeitspsychologie/gpower.html/]

The data that is going to be collected includes the following characteristics per patient:

### 1. Medical Record Number

### 2. Age

### 3. Sex

### 4. Ethnicity

### 5. Date of Birth

6. Date of admission
7. Date of discharge, or death (if applicable)
8. Selenium levels
9. Oxygen saturation
10. Alanine transaminase (ALT) levels
11. Aspartate transaminase (AST) levels
12. Creatinine levels
13. Glucose levels
14. Hemoglobin levels
15. Platelets levels
16. Prothrombin time (PT)
17. C-Reactive Protein (CRP) levels
18. Ferritin levels
19. D-dimer levels
20. Total bilirubin levels
21. White Blood Cell counts (WBC) with differential

### 22. Complete Blood Counts (CBC)

23. Interleukin-1 levels
24. Interleukin-6 levels
25. TNF-alpha levels
26. SARS CoV-2 Polymerase Chain Reaction (PCR) test results
27. All medications prior to and during hospitalization
28. Ventilator status and settings, if applicable
29. Supplemental oxygen status
30. Adverse events

### 31. Co-morbidities

The aforementioned data will be collected via Chart review. The tool used to gather the data will primarily be an Excel spreadsheet. Principal investigator and co-investigators will be responsible for data collection. Only the medical records of research subjects who have signed consent documents will be viewed for the research study. Any data that will be analyzed for bioinformatics and biostatistics will be de-identified.

The collected data would then be analyzed by a biostatistician, the principal investigator, and co-investigators. Computer packages that would be used in the data entry and analysis may include MS Excel, SPSS, and GraphPad Prism. Other packages might be used and will be promptly reported. Types of statistical tests to be used for analysis will include t-test for qualitative variables, χ2 tests and Fisher exact tests, univariate and multivariate logistic regressions.

Medical records of research subjects who have signed consent documents will be viewed for the research study. Clinical and demographic data including name, age, gender, ethnicity, and past and present clinical history and therapy, and clinical laboratory tests will be recorded. Any data that will be analyzed for bioinformatics and biostatistics will be de-identified.

The trial is designed as a double-blinded study. Neither the participants nor investigators will be aware of participants assigned to any of the study groups. All participants will have an equal chance of being selected to the intervention arm. All patients will receive the standard of care treatment, hence, none of the subjects will be deprived of any available optimal treatment.

Risks associated with the study intervention include failure to improve or worsening of patients' conditions, and adverse events associated with selenium. Side effects of selenium are minimal. Possible side effects include muscle tenderness, tremors, lightheadedness, and facial flushing, and bleeding tendency.

The dose proposed in this clinical trial is much lower than known toxic doses. The safety of selenium at the proposed dosing has been proven in various studies (refer to reference number 30, 31, 33, 36, and 65). Nevertheless, acute poisoning typically involves a single dose that rapidly produces symptoms within minutes to hours. Selenium toxicity is determined based on the patient's clinical picture and not by laboratory values. Signs of acute toxicity include hypotension and tachycardia. Electrocardiogram abnormalities include T-wave flattening and inversion, and a prolonged QT interval. Nausea, vomiting, diarrhea, and abdominal pain are often present, and pulmonary edema can be a serious complication. Neurological symptoms include tremors, muscle spasms, irritability, confusion, delirium, and coma. Death is preceded by refractory hypotension from peripheral vasodilatation and direct myocardial depression (refer to reference number 68, and 69).

The parameters as mentioned above shall be measured starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, CRP levels, IL-1 levels, IL-6 levels, and TNF-α are measured on day 1, day 14, and day 29, and wherein the Selenium levels are measured on day 1 and day 29, starting from day 1 of the initial bolus dose administration phase.

In this example as discussed in the experimental protocol presented above, in order to evaluate the safety and effectiveness of the use of Selenium as an oral treatment option for novel coronavirus SARS-CoV-2 infection, patients with COVID-19 who exhibit the signs and symptoms of mild to moderate COVID-19 and will receive Selenium infusion for 14 days. This experiment is designed to demonstrate that Selenium treatment would decrease the death rates and increase the rate of hospital discharges among hospitalized COVID-19 patients.

In order to establish that, help would be taken by measuring and analyzing both primary and secondary clinical outcome measurements. The primary outcome to be measured is the rate of hospital discharges or deaths and looks at patients to be followed from admission until hospital discharge, or death.

The secondary outcomes to be measured would evaluate the following parameters:

1. Change from baseline in alanine transaminase (ALT) [Time Frame: Day 1 through Day 29]
2. Change from baseline in aspartate transaminase (AST) [Time Frame: Day 1 through Day 29]
3. Change from baseline in creatinine [Time Frame: Day 1 through Day 29]
4. Change from baseline in glucose [Time Frame: Day 1 through Day 29]
5. Change from baseline in hemoglobin [Time Frame: Day 1 through Day 29]
6. Change from baseline in platelets [Time Frame: Day 1 through Day 29]
7. Change from baseline in prothrombin time (PT) [Time Frame: Day 1 through Day 29]
8. Change from baseline in total bilirubin [Time Frame: Day 1 through Day 29]
9. Change from baseline in white blood cell count (WBC) with differential [Time Frame: Day 1 through Day 29]
10. Change in National Early Warning Score (NEWS) from baseline [Time Frame: Day 1 through Day 29]
The NEW score has demonstrated an ability to discriminate patients at risk of poor outcomes. This score is based on 7 clinical parameters (respiration rate, oxygen saturation, any supplemental oxygen, temperature, systolic blood pressure, heart rate, level of consciousness).
The NEW Score is being used as an efficacy measure.
11. Clinical status using ordinal scale [Time Frame: Day 1 through Day 29]
The ordinal scale is an assessment of the clinical status at the first assessment of a given study day. The scale is as follows: 1) Death: 2) Hospitalized, on invasive mechanical ventilation: 3) Hospitalized, on noninvasive ventilation or high flow oxygen devices; 4) Hospitalized, requiring supplemental oxygen: 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.
12. Cumulative incidence of serious adverse events (SAEs) [Time Frame: Day 1 through Day 29]
An SAE is defined as an AE or suspected adverse reaction is considered serious if, in the view of either the investigator, it results in death, a life-threatening AE, inpatient hospitalization or prolongation of existing hospitalization, a persistent or significant incapacity or substantial disruption of the ability to conduct normal life functions.
13. Discontinuation or temporary suspension of investigational therapeutics [Time Frame: Day 1 through Day 14]
For any reason.
14. Duration of hospitalization [Time Frame: Day 1 through Day 29]
Measured in days.
15. Duration of new non-invasive ventilation or high flow oxygen use [Time Frame: Day 1 through Day 29]
Measured in days.
16. Duration of new oxygen use [Time Frame: Day 1 through Day 29]
Measured in days.
17. Duration of new ventilator use [Time Frame: Day 1 through Day 29]
Measured in days.
18. Incidence of new non-invasive ventilation or high flow oxygen use [Time Frame: Day 1 through Day 29]
19. Incidence of new oxygen use [Time Frame: Day 1 through Day 29]
20. Incidence of new ventilator use [Time Frame: Day 1 through Day 29]
21. Mean change in the ordinal scale [Time Frame: Day 1 through Day 29]
The ordinal scale is an assessment of the clinical status at the first assessment of a given study day. The scale is as follows: 1) Death; 2) Hospitalized, on invasive mechanical ventilation; 3) Hospitalized, on noninvasive ventilation or high flow oxygen devices; 4) Hospitalized, requiring supplemental oxygen: 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.
22. Time to an improvement of one category using an ordinal scale [Time Frame. Day 1 through Day 29]
The ordinal scale is an assessment of the clinical status at the first assessment of a given study day. The scale is as follows: 1) Death: 2) Hospitalized, on invasive mechanical ventilation: 3) Hospitalized, on noninvasive ventilation or high flow oxygen devices; 4) Hospitalized, requiring supplemental oxygen: 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

### Example 3—Oral Selenium for Prevention of Disease Caused by SARS-COV-2 Infection and COVID-19

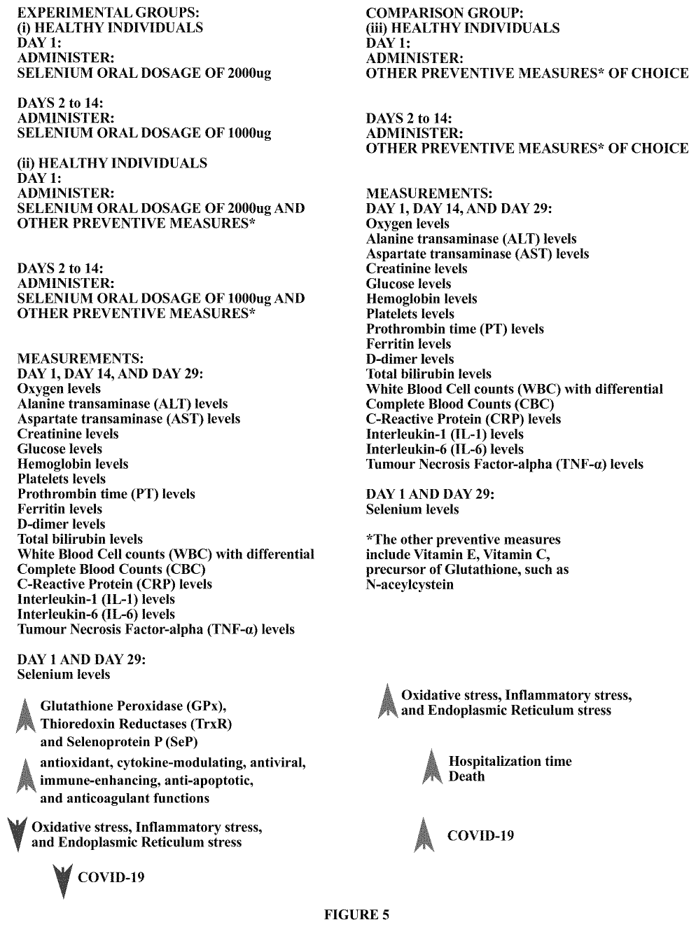

In addition to the above treatment methods, an experimental clinical trial to evaluate the potential for prevention of disease caused by SARS-COV-2 infection and COVID-19 by recruiting healthy subjects and providing them with oral dosage of Selenium as devised in the experimental design of Table 3 hereinbelow and in FIG. 5 showing the experimental design and expected results to show safety and efficacy of the proposed treatment.

As shown in the Table 3 below the experimental design envisioned for the trial is as follows:

TABLE 3
Experimental design for the experimetal group of healthy individuals to be
administeted with an intervention/prevention measure comprising a pharmacologically
acceptable molecule containing Selenium (Se) with or without other
preventative measures and the comparison group of healthy individuals who would
be administered with other preventative measures along with a placebo with regards
to the pharmacologically acceptable molecule containing Se as described below.

| Group | Intervention/treatment |
|---|---|
| (v) Experimental Group | Drug: Selenium as Selenious Acid |
| 1: to be administered | (AMERICAN REGENT ™) |
| with Selenious Add | Administered as an oral dosage as an over the |
| ONLY | counter (OTC) medication. |
| Participants who are otherwise | |
| healthy would be administered | |
| with Selenium oral dosage of | |
| 2000 μg on day 1 as a loading | |
| dose, followed by a continuous | |
| oral dosage of Selenium at a | |
| maintenance dose of 000 μg | |
| daily on days 2-14. | |
| (ii) Experimental Group 2: to be | Drug: Selenium as Selenious Acid |
| administered with Selenious Acid | (AMERICAN REGENT ™) |
| and other preventive measures | Administered as an oral dosage as an over the |
| | counter (OTC) medication. |
| Participants who are otherwise | Drug: Other preventive measures include Vitamin |
| healthy would be administered | E, Vitamin C, a precursor of Glutathione, such as |
| with Selenium oral dosage of | N-acetylcysteine, etc. |
| 2000 μg on day I as a loading | |
| dose, followed by a continuous | |
| oral dosage of Selenium at a | |
| maintenance dose of 1000 μg | |
| daily on days 2-14 along with | |
| their choice of other preventive | |
| measures. | |
| (iii) Comparison Group: to be | Drug: Other preventive measures such as Vitamin E, |
| administered Other preventive | optionally combined with vitamin C, a or |
| measures | precursor of Glutathione, such as N-acetylcysteine |
| Participants who are otherwise | |
| healthy would be orally | |
| administered other preventive | |
| measures of their choice and | |
| preference. | |

The study is planned for a duration of four months where, the first three months would include patient enrollment and data collection, one month for data analysis and publishing. Once an otherwise healthy individual meets the inclusion criteria is selected, and their consent is obtained, they will be randomly assigned to a study group and will receive the treatment regimen accordingly. They will be monitored over the following 29 days or until discharge or death.

The study is designed keeping in mind the various aspects for selection of the study subjects as follows:

Recruitment: Healthy individuals would be called to participate as subjects for this trial and will be recruited by the principal investigator, co-investigators, and delegated clinical staff at CHRISTUS Good Shepherd Medical Center. This study is completely voluntary and subjects are free to withdraw from the research at any time.

The maximum number of study subjects: 100 patients. This number includes anticipated screening and consent failures.

Inclusion Criteria:

a) Willing and able to provide written informed consent, or with a legal representative who can provide informed consent, or enrolled under International Conference on Harmonization (ICH) E6(R2) 4.8.15 emergency use provisions as deemed necessary by the investigator (age ≥18) prior to performing study procedures
b) Aged ≥18 years
c) Tested negative for Severe Acute Respiratory Syndrome Coronavirus (SARS-CoV)-2 infection by polymerase chain reaction (PCR) test <4 days before randomization

Exclusion Criteria:

a) Participation in any other clinical trial of an experimental treatment
b) Not otherwise healthy based on measured clinical parameters on day 1 of this study

It is expected that based on clinical conditions, other protocol defined Inclusion/Exclusion criteria may apply. Patient consent would be needed for participation in this study. Spanish-language consents will be made available.

For sample size calculation a Power analysis for a paired sample t-test was conducted in G*Power to determine a sufficient sample size using an alpha of 0.05, a power of 0.80, a medium effect size (dz=0.5), and two tails (Faul et al., 2013). Based on the aforementioned assumptions, a sample size of 34 participants in the experimental arm and 34 participants in the standard of care arm are needed to determine that selenium acts as a therapeutic agent for moderate, severe, and critical COVID-19 infections. [Faul, F., Erdfelder, E., Buchner, A., & Lang, A.-G. (2013). G*Power Version 3.1.9.7 [computer software]. Uiversitat Kiel. Germany. Retrieved from http://www.psychologie.hhu.de/arbeitsgruppen/allgemeine-psychologie-und-arbeitspsychologie/gpower.html/]

The data that is going to be collected includes the following characteristics per patient:

### 1. Medical Record Number

### 2. Age

### 3. Sex

### 4. Ethnicity

### 5. Date of Birth

6. Date of admission
7. Date of discharge, or death (if applicable)
8. Selenium levels
9. Oxygen saturation
10. Alanine transaminase (ALT) levels
11. Aspartate transaminase (AST) levels
12. Creatinine levels
13. Glucose levels
14. Hemoglobin levels
15. Platelets levels
16. Prothrombin time (PT)
17. C-Reactive Protein (CRP) levels
18. Ferritin levels
19. D-dimer levels
20. Total bilirubin levels
21. White Blood Cell counts (WBC) with differential

### 22. Complete Blood Counts (CBC)

23. Interleukin-1 levels
24. Interleukin-6 levels
25. TNF-alpha levels
26. SARS CoV-2 Polymerase Chain Reaction (PCR) test results
27. All medications prior to and during hospitalization
28. Ventilator status and settings, if applicable
29. Supplemental oxygen status
30. Adverse events

### 31. Co-morbidities

The aforementioned data will be collected via Chart review. The tool used to gather the data will primarily be an Excel spreadsheet. Principal investigator and co-investigators will be responsible for data collection. Only the medical records of research subjects who have signed consent documents will be viewed for the research study. Any data that will be analyzed for bioinformatics and biostatistics will be de-identified.

The collected data would then be analyzed by a biostatistician, the principal investigator, and co-investigators. Computer packages that would be used in the data entry and analysis may include MS Excel, SPSS, and GraphPad Prism. Other packages might be used and will be promptly reported. Types of statistical tests to be used for analysis will include t-test for qualitative variables, χ2 tests and Fisher exact tests, univariate and multivariate logistic regressions.

Medical records of research subjects who have signed consent documents will be viewed for the research study. Clinical and demographic data including name, age, gender, ethnicity, and past and present clinical history and therapy, and clinical laboratory tests will be recorded. Any data that will be analyzed for bioinformatics and biostatistics will be de-identified.

The trial is designed as a double-blinded study. Neither the participants nor investigators will be aware of participants assigned to any of the study groups. All participants will have an equal chance of being selected to the intervention arm. All patients will receive the standard of care treatment, hence, none of the subjects will be deprived of any available optimal treatment.

Risks associated with the study intervention include failure to improve or worsening of patients' conditions, and adverse events associated with selenium. Side effects of selenium are minimal. Possible side effects include muscle tenderness, tremors, lightheadedness, and facial flushing, and bleeding tendency.

The dose proposed in this clinical trial is much lower than known toxic doses. The safety of selenium at the proposed dosing has been proven in various studies (refer to reference number 30, 31, 33, 36, and 65). Nevertheless, acute poisoning typically involves a single dose that rapidly produces symptoms within minutes to hours. Selenium toxicity is determined based on the patient's clinical picture and not by laboratory values. Signs of acute toxicity include hypotension and tachycardia. Electrocardiogram abnormalities include T-wave flattening and inversion, and a prolonged QT interval. Nausea, vomiting, diarrhea, and abdominal pain are often present, and pulmonary edema can be a serious complication. Neurological symptoms include tremors, muscle spasms, irritability, confusion, delirium, and coma. Death is preceded by refractory hypotension from peripheral vasodilatation and direct myocardial depression (refer to reference number 68, and 69).

The parameters as mentioned above shall be measured starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, CRP levels, IL-1 levels, IL-6 levels, and TNF-α are measured on day 1, day 14, and day 29, and wherein the Selenium levels are measured on day 1 and day 29, starting from day 1 of the initial bolus dose administration phase.

In this example as discussed in the experimental protocol presented above, in order to evaluate the safety and effectiveness of the use of Selenium as an over the counter (OTC) medication as an oral preventative option for combating infections caused by novel coronavirus SARS-CoV-2 infection in otherwise healthy individuals who will receive Selenium infusion for 14 days. This experiment is designed to demonstrate that Selenium could be effective in a method of prevention strategy against SARS-CoV-2 infections and COVID-19 as an OTC medication.

In order to establish that, help would be taken by measuring and analyzing both primary and secondary clinical outcome measurements. The primary outcome to be measured is the rate of hospital discharges or deaths and looks at patients to be followed from admission until hospital discharge, or death.

The secondary outcomes to be measured would evaluate the following parameters:

1. Change from baseline in alanine transaminase (ALT) [Time Frame: Day 1 through Day 29]
2. Change from baseline in aspartate transaminase (AST) [Time Frame: Day 1 through Day 29]
3. Change from baseline in creatinine [Time Frame: Day 1 through Day 29]
4. Change from baseline in glucose [Time Frame: Day 1 through Day 29]
5. Change from baseline in hemoglobin [Time Frame: Day 1 through Day 29]
6. Change from baseline in platelets [Time Frame: Day 1 through Day 29]
7. Change from baseline in prothrombin time (PT) [Time Frame: Day 1 through Day 29]
8. Change from baseline in total bilirubin [Time Frame: Day 1 through Day 29]
9. Change from baseline in white blood cell count (WBC) with differential [Time Frame: Day 1 through Day 29]
10. Change in National Early Warning Score (NEWS) from baseline [Time Frame: Day 1 through Day 29]
The NEW score has demonstrated an ability to discriminate patients at risk of poor outcomes. This score is based on 7 clinical parameters (respiration rate, oxygen saturation, any supplemental oxygen, temperature, systolic blood pressure, heart rate, level of consciousness).
The NEW Score is being used as an efficacy measure.
11. Clinical status using ordinal scale [Time Frame: Day 1 through Day 29]
The ordinal scale is an assessment of the clinical status at the first assessment of a given study day. The scale is as follows: 1) Death: 2) Hospitalized, on invasive mechanical ventilation: 3) Hospitalized, on noninvasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.
12. Cumulative incidence of serious adverse events (SAEs) [Time Frame: Day 1 through Day 29]
An SAE is defined as an AE or suspected adverse reaction is considered serious if, in the view of either the investigator, it results in death, a life-threatening AE, inpatient hospitalization or prolongation of existing hospitalization, a persistent or significant incapacity or substantial disruption of the ability to conduct normal life functions.
13. Discontinuation or temporary suspension of investigational therapeutics [Time Frame: Day 1 through Day 14]
For any reason.
14. Duration of hospitalization [Time Frame: Day 1 through Day 29]
Measured in days.
15. Duration of new non-invasive ventilation or high flow oxygen use [Time Frame: Day 1 through Day 29]
Measured in days.
16. Duration of new oxygen use [Time Frame: Day 1 through Day 29]
Measured in days.
17. Duration of new ventilator use [Time Frame: Day 1 through Day 29]
Measured in days.
18. Incidence of new non-invasive ventilation or high flow oxygen use [Time Frame: Day 1 through Day 29]
19. Incidence of new oxygen use [Time Frame: Day 1 through Day 29]
20. Incidence of new ventilator use [Time Frame: Day 1 through Day 29]
21. Mean change in the ordinal scale [Time Frame: Day 1 through Day 29]
The ordinal scale is an assessment of the clinical status at the first assessment of a given study day. The scale is as follows: 1) Death; 2) Hospitalized, on invasive mechanical ventilation; 3) Hospitalized, on noninvasive ventilation or high flow oxygen devices: 4) Hospitalized, requiring supplemental oxygen: 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.
22. Time to an improvement of one category using an ordinal scale [Time Frame: Day 1 through Day 29]
The ordinal scale is an assessment of the clinical status at the first assessment of a given study day. The scale is as follows: 1) Death: 2) Hospitalized, on invasive mechanical ventilation: 3) Hospitalized, on noninvasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities and/or requiring home oxygen; 8) Not hospitalized, no limitations on activities.

### Advantages and Benefits

The present invention is advantageous in that there are no therapeutic modalities that are curative for COVID-19. Standard of care includes antibiotics (usually Azithromycin, and Ceftriaxone), steroids (Dexamethasone), and other therapeutic agents under further investigation including Convalescent Plasma transfusions. The anti-viral agent Remdesivir has been used in cases with some success. There is a dire need for widely-available and cost-effective treatment regimens given the increasing death toll in the US and worldwide. Selenium is a readily-available and cheap alternative that can be life-saving for thousands of patients. Patients participating in the study will still receive the standard of care therapeutic regimen in addition to selenium infusions and hence, gain the benefit of the potential effectiveness of selenium without affecting other treatment courses. Patients' condition could therefore possibly improve with selenium treatment.

Furthermore, the study would help us further understand the pathophysiological basis of COVID-19 through the efficacy of selenium. Since selenium acts through various pathways, the study could possibly guide scientists into developing more targeted therapeutic agents that harness the mechanisms of action of selenium. Thus, as exemplified above, one aspect of the invention is to extend this study to further include patients with mild COVID-19 infections using oral selenium. If Selenium is proven efficacious, over-the-counter selenium tablets could be game-changing in our fight against the novel coronavirus disease.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

1. A method for treatment of Coronavirus Disease 19 (COVID-19) and associated clinical presentations caused by novel Severe Acute Respiratory Syndrome Coronavirus-2 (SARS-CoV-2) infection, the method comprising the steps of:
administrating to a subject, a therapeutically effective amount of at least a pharmacologically acceptable molecule containing Selenium (Se) referred to as a bolus dose in an initial bolus dose administration phase;
monitoring primary and secondary outcomes with the bolus dose;
administrating to said subject, a therapeutically effective amount of at least a pharmacologically acceptable molecule containing Se referred to as a reduced, continuous dose in a successive reduced, continuous dose administration phase; and
monitoring primary and secondary outcomes with the reduced, continuous dose,
wherein the therapeutically effective amount of the bolus dose is a daily dose in a range between 1000 μg per day up to 6000 μg per day, corresponding to blood Se levels in a range between 0.0125 mg/kg up to 0.075 mg/kg of bodyweight, administered daily as required,
wherein the therapeutically effective amount of the reduced, continuous dose is a daily dose in a range between 1000 μg per day up to 1600 μg per day, corresponding to blood Se levels in a range between 0.0125 mg/kg up to 0.0200 mg/kg of bodyweight, administered daily as required,
wherein the subject is monitored in terms of age, sex, ethnicity, Selenium levels, Oxygen levels, Alanine transaminase (ALT) levels, Aspartate transaminase (AST) levels, Creatinine levels, Glucose levels, Hemoglobin levels, Platelets levels, Prothrombin time (PT) levels, C-Reactive Protein (CRP) levels, Ferritin levels, D-dimer levels, total bilirubin levels, White Blood Cell counts (WBC) with differential levels, Complete Blood Counts (CBC) levels, Interleukin-1 (IL-1) levels, Interleukin-6 (IL-6) levels, Tumour Necrosis Factor-alpha (TNF-α) levels, SARS CoV-2 Polymerase Chain Reaction (PCR) test results, all medications prior to and during hospitalization, ventilator status and settings, as required, supplemental oxygen status, adverse events, and co-morbidities, and
wherein the subject is a human or a subject under veterinary medicine.

2. The method of claim 1, wherein the at least a pharmacologically acceptable molecule containing Selenium (Se) is selected from the group consisting of:
a selenium hydride of the formula Seₓ H_{y}, wherein x is an integer from 1 to 10 and y has the same value than x;
a selenium salt selected from the group consisting of a fluorine salt of selenium, a chlorine salt of selenium, a bromine salt of selenium, an iodine salt of selenium, a selenium oxide, a sulphur salt of selenium, a tellurium salt of selenium a potassium salt of selenium, a sodium salt of selenium, a copper salt of selenium, a germanium salt of selenium, a barium salt of selenium, a lead salt of selenium, a zinc salt of selenium, or a nitrogen salt of selenium, particularly sodium selenite;
an inorganic selenium salt including a selenite, selenate or selenide selected from the group consisting of Antimony (III) selenide [Sb₂Se₃], Arsenic (III) selenide [As₂Se₃], Bismuth (III) selenide [Bi2Se3], Cadmium selenide [CdSe], Cobalt (II) selenide [CoSe], Mercury (II) selenide [HgSe], Selenium oxychloride, Seleninyl chloride [Cl₂Ose], Selenium sulfide, Selenium disulfide [S₂Se], Silver (I) selenide [Ag₂Se], Indium (III) selenide [In₂Se₃] and Strontium selenide [SeSr];
a selenium compound selected from the group consisting of Selenic acid [H₂O₄Se], Selenium dioxide [O₂Se], Selenium [Se]; Selenous acid and Selenious acid [H₂O₃Se];
an organic selenium selected from the group of selenomethionine, selenodiglutathione, selenocysteine, selenomethyl selenocysteine, dimethyl selenoxide, and selenocystamine;
a methylated derivative of selenium;
a selenium-containing amino acid;
a selenium-containing protein selected from the group consisting of a bacterial or fungal or a mammal Selenium-containing protein;
a selenium-containing organic compound selected from the group consisting of organic compounds consisting of alkyl compounds, alicyclic compounds, cyclane compounds, terpenic compounds, aromatic compounds and heterocyclic compounds; and
selenated yeasts or synthetic chemicals containing one or more atoms of Selenium.

3. The method of claim 1, wherein the bolus dose is administered in the initial bolus dose administration phase lasting between day 1 and day 3 of said bolus dose administration, administered daily as required.

4. The method of claim 1, wherein the successive reduced, continuous dose administration phase lasting between day 2 and day 14 from the day of the last bolus dose administration as the two successive phases of treatment, and said reduced, continuous dose is administered daily as required, and the total administration period including the initial bolus dose administration phase and the reduced, continuous dose administration phase is 14 days.

5. The method of claim 1, wherein the administrations of the bolus dose and the reduced, continuous dose are in the form of an injectable or pharmaceutical form.

6. The method of claim 1, wherein the administrations of the bolus dose and the reduced, continuous dose are carried out by intravenous, subcutaneous, intramuscular, intraperitoneal, or enteral routes.

7. The method of claim 1, wherein the administrations of the bolus dose and the reduced, continuous dose are carried out by intravenous infusion.

8. The method of claim 1, wherein the treatment of COVID-19 includes its clinical presentations ranging from mild, to moderate, to severe including pneumonia requiring hospitalization, to critical requiring admission to the admission into the Intensive Care Unit (ICU) and mechanical ventilation.

9. The method of claim 1, wherein the associated clinical presentations include Acute Respiratory Distress Syndrome (ARDS), Severe Inflammatory Response Syndrome (SIRS), any state corresponding to a severe acute attack of an inflammatory pathology causing an exacerbation of cytokine release and recruitment of inflammatory cells, alveolar damage, surfactant abnormalities, increased alveolar capillary permeability, decreased alveolar clearance, the release of the proteinaceous fluid within the alveoli and ultimately hypoxia, Interstitial Pulmonary Fibrosis (IPF), Acute Lung Injury (ALI), cardiotoxicity, and nephrotoxicity.

10. The method of claim 1, wherein the administrations of the bolus dose and the reduced, continuous dose are carried out in addition to the Standard of Care (SOC) treatment.

11. The method of claim 1, wherein the SOC treatment is selected from the group consisting of steroids including Dexamethasone, antibiotics including Azithromycin, and Ceftriaxone, anti-viral including Remdesivir, and Convalescent Plasma or a combination thereof.

12. The method of claim 1, wherein the monitoring primary and secondary outcomes with the bolus dose in the initial bolus dose administration phase and the monitoring primary and secondary outcomes with the reduced, continuous dose in the successive reduced, continuous dose administration phase is carried out for a total period of 29 days or until discharge or death, starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, are measured daily for a total period of 29 days or until discharge or death, wherein the CRP levels, IL-1 levels, IL-6 levels, and TNF-α are measured on day 1, day 3, day 5, day 7, day 10, day 14, day 21, and day 29, and wherein the Selenium levels are measured on day 1 and day 29, starting from day 1 of the initial bolus dose administration phase.

13. The method of claim 1, wherein the monitoring primary outcomes comprises calculation of the rate of hospital discharges or deaths, wherein a subject is followed until hospital discharge, or death from the date of admission.

14. The method of claim 1, wherein the monitoring secondary outcomes comprises calculations of:
a. change from baseline in alanine transaminase (ALT) measured in a time frame of day 1 through day 29;
b. change from baseline in aspartate transaminase (AST) measured in a time frame of day 1 through day 29;
c. change from baseline in creatinine measured in a time frame of day 1 through day 29;
d. change from baseline in glucose measured in a time frame of day 1 through day 29;
e. change from baseline in hemoglobin measured in a time frame of day 1 through day 29;
f. change from baseline in platelets measured in a time frame of day 1 through day 29;
g. change from baseline in prothrombin time (PT) measured in a time frame of day 1 through day 29;
h. change from baseline in total bilirubin measured in a time frame of day 1 through day 29;
i. change from baseline in white blood cell count (WBC) with differential measured in a time frame of day 1 through day 29;
j. change in National Early Warning Score (NEWS) from baseline measured in a time frame of day 1 through day 29;
k. clinical status using ordinal scale measured in a time frame of day 1 through day 29;
l. cumulative incidence of serious adverse events (SAEs) measured in a time frame of day 1 through day 29;
m. discontinuation or temporary suspension of investigational therapeutics measured in a time frame of day 1 through day 14;
n. duration of hospitalization measured in a time frame of day 1 through day 29;
o. duration of new non-invasive ventilation or high flow oxygen use measured in a time frame of day 1 through day 29;
p. duration of new oxygen use measured in a time frame of day 1 through day 29;
q. duration of new ventilator use measured in a time frame of day 1 through day 29;
r. incidence of new non-invasive ventilation or high flow oxygen use measured in a time frame of day 1 through day 29;
s. incidence of new oxygen use measured in a time frame of day 1 through day 29;
t. incidence of new ventilator use measured in a time frame of day 1 through day 29;
u. mean change in the ordinal scale measured in a time frame of day 1 through day 29; and
v. time to an improvement of one category using an ordinal scale measured in a time frame of day 1 through day 29,
wherein day 1 is the day 1 of the initial bolus dose administration phase,
wherein the NEWS has demonstrated an ability to discriminate patients at risk of poor outcomes and it is based on 7 clinical parameters consisting of respiration rate, oxygen saturation, any supplemental oxygen, temperature, systolic blood pressure, heart rate, level of consciousness,
wherein the ordinal scale is an assessment of the clinical status at the first assessment of a given study day and is provided as a scale ranging from: i) death; ii) hospitalized, on invasive mechanical ventilation; iii) hospitalized, on non-invasive ventilation or high flow oxygen devices; iv) hospitalized, requiring supplemental oxygen; v) hospitalized, not requiring supplemental oxygen—requiring ongoing medical care (COVID-19 related or otherwise); vi) hospitalized, not requiring supplemental oxygen—no longer requires ongoing medical care; vii) not hospitalized, limitation on activities and/or requiring home oxygen; and viii) not hospitalized, no limitations on activities, and
wherein the SAEs are defined in terms of an adverse event (AE) or suspected adverse reaction when considered serious in the view of either the investigator, it results in death, a life-threatening AE, inpatient hospitalization or prolongation of existing hospitalization, a persistent or significant incapacity or substantial disruption of the ability to conduct normal life functions.

15. The method of claim 1, wherein the bolus dose in an initial bolus dose administration phase, or the reduced, continuous dose in a successive reduced, continuous dose administration phase, or both the bolus dose in an initial bolus dose administration phase, the reduced, continuous dose in a successive reduced, continuous dose administration phase, is in combination with a therapeutically effective quantity of at least a non-selenium compound, wherein the non-selenium compound is capable of inhibiting oxidative metabolism or acting against the consequences of oxidative stress or inhibiting the inflammatory reaction or exhibiting antiviral properties or exhibiting antiapoptotic properties or a combination thereof.

16. A method for prevention or treatment of COVID-19 and associated clinical presentations caused by novel SARS-CoV-2 infection, the method comprising the steps of:
administrating to a subject, a therapeutically effective amount of at least a pharmacologically acceptable molecule containing Selenium (Se) referred to as a bolus dose in an initial bolus dose administration phase;
monitoring clinical outcomes with the bolus dose;
administrating to said subject, a therapeutically effective amount of at least a pharmacologically acceptable molecule containing Se referred to as a reduced, continuous dose in a successive reduced, continuous dose administration phase; and
monitoring clinical outcomes with the reduced, continuous dose,
wherein the therapeutically effective amount of the bolus dose is a daily dose in a range between 1000 μg per day up to 6000 μg per day, corresponding to blood Se levels in a range between 0.0125 mg/kg up to 0.075 mg/kg of bodyweight, administered daily as required,
wherein the therapeutically effective amount of the reduced, continuous dose is a daily dose in a range between 1000 μg per day up to 1600 μg per day, corresponding to blood Se levels in a range between 0.0125 mg/kg up to 0.0200 mg/kg of bodyweight, administered daily as required,
wherein the subject is monitored in terms of age, sex, ethnicity, Selenium levels, Oxygen levels, Alanine transaminase (ALT) levels, Aspartate transaminase (AST) levels, Creatinine levels, Glucose levels, Hemoglobin levels, Platelets levels, Prothrombin time (PT), C-Reactive Protein (CRP) levels, Ferritin levels, D-dimer levels, total bilirubin levels, White Blood Cell counts (WBC) with differential, Complete Blood Counts (CBC), Interleukin-1 (IL-1) levels, Interleukin-6 (IL-6) levels, Tumour Necrosis Factor-alpha (TNF-α) levels, SARS CoV-2 Polymerase Chain Reaction (PCR) test results, all other medications prescribed and consumed by said subject, adverse events, and co-morbidities, and
wherein the subject is a human or a subject under veterinary medicine.

17. The method of claim 16, wherein the administrations of the bolus dose and the reduced, continuous dose are carried out by oral route or through a feeding tube, wherein the monitoring primary and secondary outcomes with the bolus dose in the initial bolus dose administration phase and the monitoring primary and secondary outcomes with the reduced, continuous dose in the successive reduced, continuous dose administration phase is carried out for a total period of 29 days or until discharge or death, starting from day 1 of the initial bolus dose administration phase, wherein the Oxygen levels, ALT levels, AST levels, Creatinine levels, Glucose levels, Hemoglobin levels, Platelets levels, PT, Ferritin levels, D-dimer levels, total bilirubin levels, WBC with differential levels, CBC levels, CRP levels, IL-1 levels, IL-6 levels, and TNF-α are measured on day 1, day 14, and day 29, and wherein the Selenium levels are measured on day 1 and day 29, starting from day 1 of the initial bolus dose administration phase.

18. The method of claim 16, wherein the at least a pharmacologically acceptable molecule containing Selenium (Se) is selected from the group consisting of:
a selenium hydride of the formula Seₓ H_{y}, wherein x is an integer from 1 to 10 and y has the same value than x;
a selenium salt selected from the group consisting of a fluorine salt of selenium, a chlorine salt of selenium, a bromine salt of selenium, an iodine salt of selenium, a selenium oxide, a sulphur salt of selenium, a tellurium salt of selenium a potassium salt of selenium, a sodium salt of selenium, a copper salt of selenium, a germanium salt of selenium, a barium salt of selenium, a lead salt of selenium, a zinc salt of selenium, or a nitrogen salt of selenium;
an inorganic selenium salt including a selenite, selenate or selenide selected from the group consisting of Antimony (III) selenide [Sb₂Se₃], Arsenic (III) selenide [As₂Se₃], Bismuth (III) selenide [Bi2Se3], Cadmium selenide [CdSe], Cobalt (II) selenide [CoSe], Mercury (II) selenide [HgSe], Selenium oxychloride, Seleninyl chloride [Cl₂Ose], Selenium sulfide, Selenium disulfide [S₂Se], Silver (I) selenide [Ag₂Se], Indium (III) selenide [In₂Se₃] and Strontium selenide [SeSr];
a selenium compound selected from the group consisting of Selenic acid [H₂O₄Se], Selenium dioxide [O₂Se], Selenium [Se]; Selenous acid and Selenious acid [H₂O₃Se];
an organic selenium selected from the group of selenomethionine, selenodiglutathione, selenocysteine, selenomethyl selenocysteine, dimethyl selenoxide, and selenocystamine;
a methylated derivative of selenium;
a selenium-containing amino acid;
a selenium-containing protein selected from the group consisting of a bacterial or fungal or a mammal Selenium-containing protein;
a selenium-containing organic compound selected from the group consisting of organic compounds consisting of alkyl compounds, alicyclic compounds, cyclane compounds, terpenic compounds, aromatic compounds and heterocyclic compounds; and
selenated yeasts or synthetic chemicals containing one or more atoms of Selenium.

19. The method of claim 16, wherein the monitoring the clinical outcomes comprises calculations of:
a. change from baseline in alanine transaminase (ALT) measured in a time frame of day 1 through day 29;
b. change from baseline in aspartate transaminase (AST) measured in a time frame of day 1 through day 29;
c. change from baseline in creatinine measured in a time frame of day 1 through day 29;
d. change from baseline in glucose measured in a time frame of day 1 through day 29;
e. change from baseline in hemoglobin measured in a time frame of day 1 through day 29;
f. change from baseline in platelets measured in a time frame of day 1 through day 29;
g. change from baseline in prothrombin time (PT) measured in a time frame of day 1 through day 29;
h. change from baseline in total bilirubin measured in a time frame of day 1 through day 29;
i. change from baseline in white blood cell count (WBC) with differential measured in a time frame of day 1 through day 29;
j. change in National Early Warning Score (NEWS) from baseline measured in a time frame of day 1 through day 29;
k. clinical status using ordinal scale measured in a time frame of day 1 through day 29;
l. cumulative incidence of serious adverse events (SAEs) measured in a time frame of day 1 through day 29;
m. discontinuation or temporary suspension of investigational therapeutics measured in a time frame of day 1 through day 14;
n. duration of hospitalization measured in a time frame of day 1 through day 29;
o. duration of new non-invasive ventilation or high flow oxygen use measured in a time frame of day 1 through day 29;
p. duration of new oxygen use measured in a time frame of day 1 through day 29;
q. duration of new ventilator use measured in a time frame of day 1 through day 29;
r. incidence of new non-invasive ventilation or high flow oxygen use measured in a time frame of day 1 through day 29;
s. incidence of new oxygen use measured in a time frame of day 1 through day 29;
t. incidence of new ventilator use measured in a time frame of day 1 through day 29;
u. mean change in the ordinal scale measured in a time frame of day 1 through day 29; and
v. time to an improvement of one category using an ordinal scale measured in a time frame of day 1 through day 29,
wherein day 1 is the day 1 of the initial bolus dose administration phase,
wherein the NEWS has demonstrated an ability to discriminate patients at risk of poor outcomes and it is based on 7 clinical parameters consisting of respiration rate, oxygen saturation, any supplemental oxygen, temperature, systolic blood pressure, heart rate, level of consciousness,
wherein the ordinal scale is an assessment of the clinical status at the first assessment of a given study day and is provided as a scale ranging from: i) death; ii) hospitalized, on invasive mechanical ventilation; iii) hospitalized, on non-invasive ventilation or high flow oxygen devices; iv) hospitalized, requiring supplemental oxygen; v) hospitalized, not requiring supplemental oxygen—requiring ongoing medical care (COVID-19 related or otherwise); vi) hospitalized, not requiring supplemental oxygen—no longer requires ongoing medical care; vii) not hospitalized, limitation on activities and/or requiring home oxygen; and viii) not hospitalized, no limitations on activities, and
wherein the SAEs are defined in terms of an adverse event (AE) or suspected adverse reaction when considered serious in the view of either the investigator, it results in death, a life-threatening AE, inpatient hospitalization or prolongation of existing hospitalization, a persistent or significant incapacity or substantial disruption of the ability to conduct normal life functions.

20. The method of claim 16, wherein the bolus dose in an initial bolus dose administration phase, or the reduced, continuous dose in a successive reduced, continuous dose administration phase, or both the bolus dose in an initial bolus dose administration phase, the reduced, continuous dose in a successive reduced, continuous dose administration phase, is in combination with a therapeutically effective quantity of at least a non-selenium compound, wherein the non-selenium compound is capable of inhibiting oxidative metabolism or acting against the consequences of oxidative stress or inhibiting the inflammatory reaction or exhibiting antiviral properties or exhibiting antiapoptotic properties or a combination thereof.
